(19) United States
(12) Patent Application Publication
(10) Pub. No.: US 2025/0109198 A1
(43) Pub. Date: Apr. 3, 2025

(54) APPROVED PRODUCTS FOR THE TREATMENT OF RELAPSED OR REFRACTORY MULTIPLE MYELOMA

(71) Applicant: Janssen Biotech, Inc., Horsham, PA (US)

(72) Inventors: Christoph Heuck, Princeton, NJ (US); Colleen Kane, Flourtown, PA (US); Marie-Anne Damiette Smit, Warmond (NL)

(21) Appl. No.: 18/797,622

(22) Filed: Aug. 8, 2024

Related U.S. Application Data
(60) Provisional application No. 63/518,524, filed on Aug. 9, 2023.
(60) Provisional application No. 63/518,530, filed on Aug. 9, 2023.
(60) Provisional application No. 63/578,280, filed on Aug. 23, 2023.
(60) Provisional application No. 63/589,055, filed on Oct. 10, 2023.
(60) Provisional application No. 63/589,061, filed on Oct. 10, 2023.

(51) Int. Cl. C07K 16/28; A61K 39/00

(52) U.S. Cl. CPC C07K 16/28; C07K 16/2809; A61K 2039/505; A61K 2039/545; C07K 2317/24; C07K 2317/31

(57) ABSTRACT

Described herein are approved products and methods of using approved products for treating relapsed or refractory multiple myeloma in a patient. Also described herein are methods of selling or offering for sale an approved product.

### CROSS-REFERENCE TO RELATED APPLICATIONS

This application claims priority to U.S. Provisional Application No. 63/518,524, which was filed on Aug. 9, 2023, U.S. Provisional Application No. 63/518,530, which was filed on Aug. 9, 2023, U.S. Provisional Application No. 63/578,280, which was filed on Aug. 23, 2023, U.S. Provisional Application No. 63/589,055, which was filed on Oct. 10, 2023, and U.S. Provisional Application No. 63/589,061, which was filed on Oct. 10, 2023, the disclosures of each of which are incorporated herein by reference in their entireties.

### SEQUENCE LISTING

The instant application contains a Sequence Listing which has been submitted electronically in XML file format and is hereby incorporated by reference in its entirety. Said XML copy, created on Aug. 5, 2024, is named 103693_004300_SL.xml and is 19,898 bytes in size.

### TECHNICAL FIELD

Disclosed herein are approved products and methods of using the approved products to treat relapsed or refractory multiple myeloma (MM).

### BACKGROUND

In the pharmaceutical industry, the path from drug discovery to reaching an approved product is riddled with uncertainty. That path is one that since 2010 and out of more than 12,000 clinical studies, there is very low likelihood of regulatory approval of less than 10% (from Phase 1 to approval). The final path to approval is also not a certainty nor inevitable as failures continue as late as from Phase 3 to approval. This is not only due to the high unpredictability and uncertainty regarding how a pharmaceutical product will impact human biology, but often human behavior is unpredictable in how data is interpreted by regulatory agencies and whether the data warrants approving a study drug as an approved product for use in commerce.

As of February 2022, the five-year survival rate for people with multiple myeloma (MM) in the United States was 55%. Unfortunately, it is estimated that in the United States more than 12,000 people will die from the disease in 2023 (cancer.net/cancer-types/multiple-myeloma/statistics).

Between the year 2000 and as of the filing of this document, there had been more than 200 molecules assessed in clinical trials with the hope of identifying a treatment for patients suffering from MM. (Pharmalntelligence, Pharmaprojects database results using terms “Cancer, myeloma”). Of those molecules, only nineteen (19) pioneer molecules had been incorporated into approved products and used to treat MM patients. Another 185 efforts to treat MM as either a lead molecule or in combination were suspended and did not progress. This data makes clear that the path to effective patient treatments for MM remains complex and unpredictable to this day. Despite the existence of the nineteen approved medicines, there remains a significant unmet patient need to treat patients with MM.

### SUMMARY

Disclosed herein are approved products comprising 3 mg/1.5 mL (2 mg/mL) of talquetamab-tgvs in a single-dose vial and/or 40 mg/mL of talquetamab-tgvs in a single-dose vial.

Disclosed herein are combinations of a corticosteroid, an antihistamine, an antipyretic, and an approved product.

Disclosed herein are pharmaceutical products comprising talquetamab-tgvs in a single-dose vial, wherein the pharmaceutical product is packaged, and wherein the package includes a label that identifies the pharmaceutical product as indicated as monotherapy for the treatment of adult patients with relapsed or refractory multiple myeloma, who have received at least four prior lines of therapy, including a proteasome inhibitor, an immunomodulatory agent, and an anti-CD38 monoclonal antibody.

Disclosed herein are kits comprising any of the disclosed approved products, any of the disclosed combinations, or any of the disclosed pharmaceutical products.

Disclosed herein are methods for treating relapsed or refractory multiple myeloma in a patient, the methods comprising administering any of the disclosed approved products or any of the disclosed combinations to the patient in an amount and manner that is described in a drug product label for the approved product.

Disclosed herein are methods for treating relapsed or refractory multiple myeloma in a patient, the methods comprising administering a corticosteroid, an antihistamine, an antipyretic, and any of the disclosed approved products to the patient in an amount and manner that is described in a drug product label for the approved product.

Disclosed herein are methods of selling an approved product, the methods comprising selling the approved product, wherein an approved product label for a reference product for the approved product includes instructions for treating adult patients with relapsed or refractory multiple myeloma after at least four prior lines of therapy, including a proteasome inhibitor, an immunomodulatory agent, and an anti-CD38 monoclonal antibody.

Disclosed herein are methods of offering for sale an approved product, the methods comprising offering for sale the approved product, wherein an approved product label for a reference product for the approved product includes instructions for treating adult patients with relapsed or refractory multiple myeloma after at least four prior lines of therapy, including a proteasome inhibitor, an immunomodulatory agent, and an anti-CD38 monoclonal antibody.

Disclosed herein are methods of negotiating a maximum fair price for an approved product, the methods comprising identifying the approved product as negotiation eligible, selecting the negotiation eligible approved product to be included in a price negotiation with a license holder of the approved product, wherein the license holder and a third party enter into negotiations to determine a maximum fair price for the negotiation eligible approved product, and concluding the negotiations with an agreed-upon maximum fair price for the negotiation eligible approved product.

### DETAILED DESCRIPTION OF ILLUSTRATIVE EMBODIMENTS

The disclosed approved products and methods may be understood more readily by reference to the following detailed description. It is to be understood that the disclosed approved products and methods are not limited to the specific approved products and methods described and/or shown herein, and that the terminology used herein is for the purpose of describing particular embodiments by way of example only and is not intended to be limiting of the claimed approved products and methods.

Unless specifically stated otherwise, any description as to a possible mechanism or mode of action or reason for improvement is meant to be illustrative only, and the disclosed approved products and methods are not to be constrained by the correctness or incorrectness of any such suggested mechanism or mode of action or reason for improvement.

Throughout this text, the descriptions refer to approved products and methods. Where the disclosure describes or claims a feature or embodiment associated with an approved product, such a feature or embodiment is equally applicable to the methods. Likewise, where the disclosure describes or claims a feature or embodiment associated with the methods, such a feature or embodiment is equally applicable to the approved products.

When a value is expressed as an approximation by use of the descriptor “about,” it will be understood that the particular value forms another embodiment. Reference to a particular numerical value includes at least that particular value, unless the context clearly dictates otherwise. In general, use of the term “about” indicates approximations that can vary depending on the desired properties sought to be obtained by the disclosed subject matter and is to be interpreted in the specific context in which it is used, based on its function. In some cases, the number of significant figures used for a particular value may be one non-limiting method of determining the extent of the word “about.” In other cases, the gradations used in a series of values may be used to determine the intended range available to the term “about” for each value.

The term “about” as used herein when referring to a measurable value such as an amount, a temporal duration, and the like, is meant to encompass variations of up to ±10% from the specified value. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the herein disclosure. Where a range of numerical values is stated herein as being less than a stated value, the range is nevertheless bounded on its lower end by a non-zero value. It is not intended that the scope of the approved products and methods be limited to the specific values recited when defining a range. All ranges are inclusive and combinable.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the disclosed approved products and methods which are, for clarity, described herein in the context of separate embodiments, may also be provided in combination in a single embodiment. Conversely, various features of the disclosed approved products and methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of:” similarly, the term “consisting essentially of” is intended to include examples encompassed by the term “consisting of”

The term “approved product” means: a pharmaceutical product that i) has been approved for introduction into commerce by a regulatory agency for use in treating humans with relapsed or refractory multiple myeloma who have received at least four prior lines of therapy, including a proteasome inhibitor, an immunomodulatory agent, and an anti-CD38 monoclonal antibody; ii) includes a biological product having at least the amino acid sequences of SEQ ID NOs: 9, 10, 19, and 20; iii) is formulated for delivery by injection in either a 2 mg/ml or 40 mg/ml concentration; and iv) induces an overall response rate (ORR) of at least 70% when administered weekly or biweekly in humans who previously received at least three prior lines of therapy, including a proteasome inhibitor, an immunomodulatory agent, and an anti-CD38 monoclonal antibody. An example of the “approved product” is the product listed in the approved product label of Example 1.

The term “biosimilar” means a product approved by a regulatory agency comprising an antibody that is highly similar to and does not cause any clinically meaningful differences when administered to patients relative to a pioneer company's approved product (reference product). A biosimilar can be shown to be highly similar to the reference product by extensively analyzing (i.e., characterizing) the structure and function of both the reference product and the proposed biosimilar and comparing characteristics of the products, such as purity, chemical identity, and bioactivity. Different oligosaccharide profiles may exist between products produced using different cell lines or manufacturing techniques and a biosimilar may behave differently in patients if manufactured using a different cell line or a different methodology.

The manufacturer of a proposed biosimilar product generates data comparing the proposed product to the reference product in order to demonstrate biosimilarity. A biosimilar product application must include data demonstrating biosimilarity to the reference product, which includes data from:
Analytical studies demonstrating that the proposed biosimilar product is highly similar to the reference product, notwithstanding minor differences in clinically inactive components;
Animal studies, including an assessment of toxicity; and
A clinical study or studies sufficient to demonstrate safety, purity, and potency of the proposed biosimilar product in one or more of the indications for which the reference product is licensed, which typically includes assessing immunogenicity, pharmacokinetics (PK), and, in some cases, pharmacodynamics (PD) and may also include a comparative clinical study.

A biosimilar manufacturer may not need to conduct as many clinical trials and can simply rely on the innovative research, development, and clinical work of the pioneer company to bring its biosimilar forward into commerce.

The term “reference product” refers to a product that is approved by a regulatory agency (e.g., the approved product) against which a proposed biosimilar product is compared. A reference product is approved based on, among other things, a full complement of safety and effectiveness data. A proposed biosimilar product is compared to, and evaluated against, a reference product to ensure that the product is highly similar and has no clinically meaningful differences.

The term “overall response rate” (ORR) means the proportion of subjects who achieved stringent complete response (sCR), complete response (CR), very good partial response (VGPR), and partial response (PR), during or after study treatment but before the start of subsequent anti-myeloma therapy. ORR was assessed using the International Myeloma Working Group (IMWG) 2016 response criteria as the primary endpoint. ORR is described further in Chari, A., et al., Talquetamab, a T-Cell-Redirecting GPRC5D Bispecific Antibody for Multiple Myeloma, N Engl J Med (2022); 387:2232-2244, which is incorporated by reference herein. The criteria for ORR as it relates to the approved product described herein are further detailed in Example 1.

“Label” or “approved product label” refers to information provided to a patient which provides relevant information regarding the approved product. Such information includes, without limitation, one or more of: the description of the approved product, clinical pharmacology, indications (uses for the approved product), contraindication (who should not take the approved product), warnings, precautions, adverse events (side effects), drug abuse and dependence, dosage and administration, use in pregnancy, use in nursing mothers, use in children and older patients, how the approved product is supplied, safety information for the patient, or any combination thereof. In some embodiments, the label or approved product label identifies talquetamab-tgvs and provides instructions for its use in a patient.

“Antibody” refers to all isotypes of immunoglobulins (IgG, IgA, IgE, IgM, IgD, and IgY) including various monomeric, polymeric, and chimeric forms, unless otherwise specified. Specifically encompassed by the term “antibody” are polyclonal antibodies, monoclonal antibodies (mAbs), and antibody-like polypeptides, such as chimeric antibodies and humanized antibodies.

“CD3” refers to the human CD3 protein multi-subunit complex. The term “CD3” includes any CD3 variant, isoform and species homolog which is naturally expressed by cells (including T cells).

“GPRC5D” refers to G protein-coupled receptor, class C group 5 member D. GPRC5D is a 7-transmembrane receptor protein that is classified as a type C G protein-coupled receptor based on the sequence homology score. GPRC5D is an orphan receptor whose ligand and signaling mechanisms are yet to be identified.

Talquetamab-tgvs is a bispecific T-cell engaging antibody that binds to the CD3 receptor expressed on the surface of T-cells and G protein-coupled receptor class C group 5 member D (GPRC5D) expressed on the surface of multiple myeloma cells and non-malignant plasma cells, and minimal expression on B cells, as well as healthy tissues such as epithelial cells in keratinized tissues of the skin and tongue. See, e.g., Chari A., et al., Blood 2022; 140 (suppl 1): 384-387, and Chari A., et al., N Engl J Med 2022; 387:2232-2244, which are incorporated by reference herein. Talquetamab-tgvs has the amino acid sequences described in Table C. Talquetamab-tgvs is prepared by an exchange of the Fab arms of the anti-GPRC5D mAb and the anti-CD3 mAb. Talquetamab-tgvs is an IgG4-like bispecific antibody comprising Fc substitutions, with the GPRC5D-binding arm and CD3-binding arm each comprising P/A/A substitutions at amino acid positions 228/234/235, respectively; and the CD3-binding arm also comprising F405L and R409K substitutions (according to EU index numbering). The Fab arm exchange is facilitated by the amino acid substitutions at positions F405L and R409K in the CD3 binding arm. The S228P substitutions stabilize the heterodimer, and Ala/Ala substitutions at amino acid positions 234 and 235 (according to EU index) prevent additional interactions with immune cells (Fc silencing).

As used herein, the term “patient” refers to a human. Subject and patient can be used interchangeably throughout this disclosure.

As used herein, the term “refractory” refers to disease which becomes non-responsive or progressive on therapy.

The phrase “pharmaceutically acceptable,” as used in connection with compositions described herein, refers to molecular entities and other ingredients of such compositions that are physiologically tolerable and do not typically produce untoward reactions when administered to a human. Preferably, the term “pharmaceutically acceptable” means approved by a regulatory agency of the federal or a state government or listed in the U.S. Pharmacopeia or other generally recognized pharmacopeia for use in mammals, and more particularly in humans.

The term “placebo” as used herein means administration of a pharmaceutical composition that does not include talquetamab-tgvs.

“Treat,” “treatment,” and like terms include reducing the severity and/or frequency of symptoms, eliminating symptoms and/or the underlying cause of the symptoms, reducing the frequency or likelihood of symptoms and/or their underlying cause, and improving or remediating damage caused, directly or indirectly, by the relapsed or refractory multiple myeloma. “Treatment” can also mean delaying or slowing of disease progression and/or prolonging survival as compared to expected survival if a subject was not receiving the treatment. Treatment includes, for example, achieving desired clinical results, such as the efficacy results provided in Example 1.

### Approved Products

Disclosed herein are approved products comprising 3 mg/1.5 mL (2 mg/mL) of talquetamab-tgvs in a single-dose vial and/or 40 mg/mL of talquetamab-tgvs in a single-dose vial. In some embodiments, the approved products have been approved for introduction into commerce by a regulatory agency for use in treating humans with relapsed or refractory multiple myeloma who have received at least four prior lines of therapy, including a proteasome inhibitor, an immunomodulatory agent and an anti-CD38 monoclonal antibody. Thus, disclosed herein are approved products comprising 3 mg/1.5 mL (2 mg/mL) of talquetamab-tgvs in a single-dose vial and/or 40 mg/mL of talquetamab-tgvs in a single-dose vial, wherein the approved products have been approved for introduction into commerce by a regulatory agency for use in treating humans with relapsed or refractory multiple myeloma who have received at least four prior lines of therapy, including a proteasome inhibitor, an immunomodulatory agent and an anti-CD38 monoclonal antibody. In some embodiments, the approved product comprises 3 mg/1.5 mL (2 mg/mL) of talquetamab-tgvs in a single-dose vial. In some embodiments, the approved product comprises 40 mg/mL of talquetamab-tgvs in a single-dose vial. In some embodiments, the approved product comprises both 3 mg/1.5 mL (2 mg/mL) of talquetamab-tgvs in a single-dose vial and 40 mg/mL of talquetamab-tgvs in a single-dose vial.

In an embodiment, the approved product is administered in a first step-up dose of 0.01 mg/kg and a second step-up dose of 0.06 mg/kg followed by 0.4 mg/kg administered 2 to 7 days after the second step-up dose and once weekly thereafter. In an embodiment, pretreatment medications are administered prior to each dose of the approved product's step-up dosing schedule, which includes the first step-up dose, the second step-up dose, and the first treatment dose as described in the table below.

In some embodiments, the first step dose of 0.01 mg/kg is administered using the 2 mg/mL of talquetamab-tgvs in a single-dose vial, the second step dose of 0.06 mg/kg is administered using the 2 mg/mL of talquetamab-tgvs in a single-dose vial, and each treatment dose of 0.4 mg/kg is administered using the 40 mg/mL of talquetamab-tgvs in a single-dose vial.

The approved product can be formulated for delivery to a human in a dosing schedule comprising:
0.01 mg/kg administered as a first step-up dose on Day 1;
0.06 mg/kg administered as a second step-up dose 2 to 7 days after the first step-up dose;
0.4 mg/kg administered as a first treatment dose 2 to 7 days after the second step-up dose; and
0.4 mg/kg administered as a subsequent treatment dose one week after the first treatment dose and once weekly thereafter.

An exemplary weekly dosing schedule is provided below:

TABLE 1
TALVEY Weekly Dosing Schedule

| Dosing schedule | Day | Dose^{a} | |
|---|---|---|---|
| Step-up dosing | Day 1 | Step-up dose 1 | 0.01 mg/kg |
| schedule | Day 4^{b} | Step-up dose 2 | 0.06 mg/kg |
| | Day 7^{b} | First treatment dose | 0.4 mg/kg |
| Weekly dosing | One week after first | Subsequent | 0.4 mg/kg |
| schedule | treatment dose and | treatment doses | once |
| | weekly thereafter^{c} | | weekly |

^{a}Based on actual body weight.
^{b}Dose may be administered between 2 to 4 days after the previous dose and may be given up to 7 days after the previous dose to allow for resolution of adverse reactions.
^{c}Maintain a minimum of 6 days between weekly doses.

The term “step-up dosing” refers to ascending concentrations of doses of the approved product administered prior to the weekly dosing schedule. The term “first step-up dose” refers to Step-up dose 1 in the above table. The term “second step-up dose” refers to Step-up dose 2 in the above table. The “subsequent treatment dose” can be administered one week after the first treatment dose and weekly thereafter. One or more subsequent treatment doses can be administered (e.g., once weekly on weeks 1, 2, 3, 4, 5, 6, 7, 8, or for more than 8 weeks after the first treatment dose).

In some embodiments, the second step-up dose is administered on Day 3, Day 4, or Day 5.

In some embodiments, the second step-up dose is administered on Day 4.

In some embodiments, the first treatment dose is administered 2 to 4 days after the second step-up dose.

In some embodiments, the first treatment dose is administered on Day 7.

The approved product can be formulated for delivery to a human in a dosing schedule comprising:
0.01 mg/kg administered as a first step-up dose on Day 1;
0.06 mg/kg administered as a second step-up dose on Day 4;
0.4 mg/kg administered as a first treatment dose on Day 7; and
0.4 mg/kg administered as a subsequent treatment dose one week after the first treatment dose and once weekly thereafter.

The approved product can be administered once weekly until disease progression or unacceptable toxicity. For example, TALVEY® is administered until disease progression or unacceptable toxicity.

In an embodiment, the approved product is administered in a first step-up dose of 0.01 mg/kg, a second step-up dose of 0.06 mg/kg, and a third step-up dose of 0.4 mg/kg, followed by 0.8 mg/kg administered 2 to 7 days after the third step-up dose and once every 12 to 14 days thereafter. In an embodiment, pretreatment medications are administered prior to each dose of the approved product's step-up dosing schedule, which includes step-up dose 1, step-up dose 2, step-up dose 3, and the first treatment dose as described in the table below.

In some embodiments, the first step dose of 0.01 mg/kg is administered using the 2 mg/mL of talquetamab-tgvs in a single-dose vial, the second step dose of 0.06 mg/kg is administered using the 2 mg/mL of talquetamab-tgvs in a single-dose vial, the third step-up dose of 0.4 mg/kg is administered using the 40 mg/mL of talquetamab-tgvs in a single-dose vial, and each treatment dose of 0.8 mg/kg is administered using the 40 mg/mL of talquetamab-tgvs in a single-dose vial.

The approved product can be formulated for delivery to a human in a dosing schedule comprising:
0.01 mg/kg administered as a first step-up dose on Day 1;
0.06 mg/kg administered as a second step-up dose 2 to 7 days after the first step-up dose;
0.4 mg/kg administered as a third step-up dose 2 to 7 days after the second step-up dose;
0.8 mg/kg administered as a first treatment dose 2 to 7 days after the third step-up dose; and
0.8 mg/kg administered as a subsequent treatment dose 12 to 14 days after the first treatment dose and once every two weeks (12 to 14 days) thereafter.

An exemplary biweekly dosing schedule is provided below:

TABLE 2
TALVEY Biweekly (Every 2 Weeks) Dosing Schedule

| Dosing schedule | Day | Dose^{a} | |
|---|---|---|---|
| Step-up dosing | Day 1 | Step-up dose 1 | 0.01 mg/kg |
| schedule | Day 4^{b} | Step-up dose 2 | 0.06 mg/kg |
| | Day 7^{b} | Step-up dose 3 | 0.4 mg/kg |
| | Day 10^{c} | First treatment dose | 0.8 mg/kg |
| Biweekly (every | Two weeks after first | Subsequent | 0.8 mg/kg |
| 2 weeks) dosing | treatment dose and | treatment doses | every |
| schedule | every 2 weeks | | 2 weeks |
| | thereafter^{d} | | |

^{a}Based on actual body weight.
^{b}Dose may be administered between 2 to 4 days after the previous dose and may be given up to 7 days after the previous dose to allow for resolution of adverse reactions.
^{c}Dose may be administered between 2 to 7 days after step-up dose 3.
^{d}Maintain a minimum of 12 days between biweekly (every 2 weeks) doses.

The term “first step-up dose” refers to Step-up dose 1 in the above table. The term “second step-up dose” refers to Step-up dose 2 in the above table. The term “third step-up dose” refers to Step-up dose 3 in the above table. The “subsequent treatment dose” can be administered 12 to 14 days after the first treatment dose and every 12 to 14 days thereafter. One or more subsequent treatment doses can be administered (e.g., biweekly on weeks 2, 4, 6, 8, 10, or for more than 10 weeks after the first treatment dose).

In some embodiments, the second step-up dose is administered on Day 3, Day 4, or Day 5.

In some embodiments, the second step-up dose is administered on Day 4.

In some embodiments, the third step-up dose is administered 2 to 4 days after the second step-up dose.

In some embodiments, the third step-up dose is administered on Day 7.

In some embodiments, the first treatment dose is administered 2 to 7 days after the third step-up dose.

In some embodiments, the first treatment dose is administered on Day 10.

The approved product can be formulated for delivery to a human in a dosing schedule comprising:
0.01 mg/kg administered as a first step-up dose on Day 1;
0.06 mg/kg administered as a second step-up dose on Day 4;
0.4 mg/kg administered as a third step-up dose on Day 7;
0.8 mg/kg administered as a first treatment dose on Day 10; and
0.8 mg/kg administered as a subsequent treatment dose 12 to 14 days after the first treatment dose and once every two weeks (12 to 14 days) thereafter.

The approved product can be administered biweekly (once every two weeks) until disease progression or unacceptable toxicity. For example, TALVEY® is administered until disease progression or unacceptable toxicity.

In some embodiments, the approved product is a biosimilar of talquetamab-tgvs.

The approved product can include the following excipients: edetate disodium; glacial acetic acid; polysorbate 20; sodium acetate; sucrose; and water for injection, USP.

The approved product can be in a 1.5 ml single-dose vial comprising 3 mg of talquetamab-tgvs, edetate disodium, glacial acetic acid, polysorbate 20, sodium acetate, sucrose, and water for injection, USP. In some embodiments, the approved product can be in a 1.5 ml single-dose vial comprising 3 mg of talquetamab-tgvs, 0.027 mg edetate disodium, 0.36 mg glacial acetic acid, 0.6 mg polysorbate 20, 1.39 mg sodium acetate, 120 mg sucrose, and Water for Injection, USP.

The approved product can be in a 1.0 ml single-dose vial comprising 40 mg of talquetamab-tgvs, edetate disodium, glacial acetic acid, polysorbate 20, sodium acetate, sucrose, and water for injection, USP. In some embodiments, the approved product can be in a 1.0 ml single-dose vial comprising 40 mg of talquetamab-tgvs, 0.018 mg edetate disodium, 0.24 mg glacial acetic acid, 0.4 mg polysorbate 20, 0.90 mg sodium acetate, 80 mg sucrose, and Water for Injection, USP.

The approved product can be formulated into any of the stable aqueous pharmaceutical compositions described in U.S. Provisional Patent App. No. 63/487,514, filed Feb. 28, 2023, which is incorporated by reference herein.

The approved products can be administered to the patient in combination with a corticosteroid, an antihistamine, and an antipyretic. In some embodiments, the approved products can be administered in combination with 16 mg or equivalent of oral or intravenous dexamethasone, 50 mg or equivalent of oral or intravenous diphenhydramine, and 650 mg to 1000 mg or equivalent of oral or intravenous acetaminophen. Also provided herein are combinations of a corticosteroid, an antihistamine, an antipyretic, and the approved products. The corticosteroid, antihistamine, and antipyretic can be administered prior to the first step-up dose, prior to the second step-up dose, prior to the third step-up dose, prior to the first treatment dose, and/or prior to each subsequent treatment dose. In some embodiments, the combination can comprise oral or intravenous dexamethasone, oral or intravenous diphenhydramine, and oral or intravenous acetaminophen. In some embodiments, the combination comprises 16 mg or equivalent of oral or intravenous dexamethasone, 50 mg or equivalent of oral or intravenous diphenhydramine, and 650 mg to 1000 mg or equivalent of oral or intravenous acetaminophen.

Following administration of the approved products or combinations, the median duration of response can be 9.5 months.

The approved products and combinations can be administered to a patient in accordance with a REMS program to reduce a risk that the patient experiences cytokine release syndrome (CRS) and/or neurologic toxicity (e.g., immune effector cell-associated neurotoxicity syndrome (ICANS)) after receiving the approved product.

The approved products can comprise a first heavy chain (HC) (SEQ ID NO: 9) and a first light chain (LC) (SEQ ID NO: 10) that pair to form a first antigen-binding site that immunospecifically binds GPRC5D, and a second HC (SEQ ID NO: 19) and a second LC (SEQ ID NO: 20) that pair to form a second antigen-binding site that immunospecifically binds CD3. A biosimilar can comprise, for example, a first HC having 95%, 96%, 97%, 98%, 99%, or 100% sequence identity to SEQ ID NO: 9, a first LC having 95%, 96%, 97%, 98%, 99%, or 100% sequence identity to SEQ ID NO: 10, a second HC having 95%, 96%, 97%, 98%, 99%, or 100% sequence identity to SEQ ID NO: 19, and a second LC having 95%, 96%, 97%, 98%, 99%, or 100% sequence identity to SEQ ID NO: 20. The antigen-binding site that binds GPRC5D comprises a heavy chain complementarity determining region 1 (HCDR1) comprising the amino acid sequence of SEQ ID NO: 1, HCDR2 comprising the amino acid sequence of SEQ ID NO: 2, and HCDR3 comprising the amino acid sequence of SEQ ID NO: 3, and a light chain complementarity determining region 1 (LCDR1) comprising the amino acid sequence of SEQ ID NO: 4, LCDR2 comprising the amino acid sequence of SEQ ID NO: 5, and LCDR3 comprising the amino acid sequence of SEQ ID NO: 6. The antigen-binding site that binds CD3 comprises a HCDR1 comprising the amino acid sequence of SEQ ID NO: 11, HCDR2 comprising the amino acid sequence of SEQ ID NO: 12, and HCDR3 comprising the amino acid sequence of SEQ ID NO: 13, and a LCDR1 comprising the amino acid sequence of SEQ ID NO: 14, LCDR2 comprising the amino acid sequence of SEQ ID NO: 15, and LCDR3 comprising the amino acid sequence of SEQ ID NO: 16. Antibody sequences are provided in the below table:

TABLE C
Sequences

| | | SEQ |
|---|---|---|
| | | ID |
| Name | SEQUENCE | NO: |
| GPRC5D | GYTMN | 1 |
| HCDR1 | | |
| GPRC5D | LINPYNSDTNYAQKLQG | 2 |
| HCDR2 | | |
| GPRC5D | VALRVALDY | 3 |
| HCDR3 | | |
| GPRC5D | KASQNVATHVG | 4 |
| LCDR1 | | |
| GPRC5D | SASYRYS | 5 |
| LCDR2 | | |
| GPRC5D | QQYNRYPYT | 6 |
| LCDR3 | | |
| GPRC5D VH | QVQLVQSGAEVKKPGASVKVSCKASGYSFTGYT | 7 |
| | MNWVRQAPGQGLEWMGLINPYNSDTNYAQKL | |
| | QGRVTMTTDTSTSTAYMELRSLRSDDTAVYYCA | |
| | RVALRVALDYWGQGTLVTVSS | |
| GPRC5D VL | DIQMTQSPSSLSASVGDRVTITCKASQNVATHVG | 8 |
| | WYQQKPGKAPKRLIYSASYRYSGVPSRFSGSGSG | |
| | TEFTLTISNLQPEDFATYYCQQYNRYPYTFGQGT | |
| | KLEIK | |
| GPRC5D HC | QVQLVQSGAEVKKPGASVKVSCKASGYSFTGYT | 9 |
| | MNWVRQAPGQGLEWMGLINPYNSDTNYAQKL | |
| | QGRVTMTTDTSTSTAYMELRSLRSDDTAVYYCA | |
| | RVALRVALDYWGQGTLVTVSSASTKGPSVFPLA | |
| | PCSRSTSESTAALGCLVKDYFPEPVTVSWNSGAL | |
| | TSGVHTFPAVLQSSGLYSLSSVVTVPSSSLGTKTY | |
| | TCNVDHKPSNTKVDKRVESKYGPPCPPCPAPEA | |
| | AGGPSVFLFPPKPKDTLMISRTPEVTCVVVDVSQ | |
| | EDPEVQFNWYVDGVEVHNAKTKPREEQFNSTYR | |
| | VVSVLTVLHQDWLNGKEYKCKVSNKGLPSSIEK | |
| | TISKAKGQPREPQVYTLPPSQEEMTKNQVSLTCL | |
| | VKGFYPSDIAVEWESNGQPENNYKTTPPVLDSD | |
| | GSFFLYSRLTVDKSRWQEGNVFSCSVMHEALHN | |
| | HYTQKSLSLSLGK | |
| GPRC5D LC | DIQMTQSPSSLSASVGDRVTITCKASQNVATHVG | 10 |
| | WYQQKPGKAPKRLIYSASYRYSGVPSRFSGSGSG | |
| | TEFTLTISNLQPEDFATYYCQQYNRYPYTFGQGT | |
| | KLEIKRTVAAPSVFIFPPSDEQLKSGTASVVCLLN | |
| | NFYPREAKVQWKVDNALQSGNSQESVTEQDSK | |
| | DSTYSLSSTLTLSKADYEKHKVYACEVTHQGLSS | |
| | PVTKSFNRGEC | |
| CD3 HCDR1 | TYAMN | 11 |
| CD3 HCDR2 | RIRSKYNNYATYYAASVKG | 12 |
| CD3 HCDR3 | HGNFGNSYVSWFAY | 13 |
| CD3 LCDR1 | RSSTGAVTTSNYAN | 14 |
| CD3 LCDR2 | GTNKRAP | 15 |
| CD3 LCDR3 | ALWYSNLWV | 16 |
| CD3 VH | EVQLVESGGGLVQPGGSLRLSCAASGFTFNTYA | 17 |
| | MNWVRQAPGKGLEWVARIRSKYNNYATYYAAS | |
| | VKGRFTISRDDSKNSLYLQMNSLKTEDTAVYYC | |
| | ARHGNFGNSYVSWFAYWGQGTLVTVSS | |
| CD3 VL | QTVVTQEPSLTVSPGGTVTLTCRSSTGAVTTSNY | 18 |
| | ANWVQQKPGQAPRGLIGGTNKRAPGTPARFSGS | |
| | LLGGKAALTLSGVQPEDEAEYYCALWYSNLWV | |
| | FGGGTKLTVLGQP | |
| CD3 HC | EVQLVESGGGLVQPGGSLRLSCAASGFTFNTYA | 19 |
| | MNWVRQAPGKGLEWVARIRSKYNNYATYYAAS | |
| | VKGRFTISRDDSKNSLYLQMNSLKTEDTAVYYC | |
| | ARHGNFGNSYVSWFAYWGQGTLVTVSSASTKG | |
| | PSVFPLAPCSRSTSESTAALGCLVKDYFPEPVTVS | |
| | WNSGALTSGVHTFPAVLQSSGLYSLSSVVTVPSS | |
| | SLGTKTYTCNVDHKPSNTKVDKRVESKYGPPCP | |
| | PCPAPEAAGGPSVFLFPPKPKDTLMISRTPEVTCV | |
| | VVDVSQEDPEVQFNWYVDGVEVHNAKTKPREE | |
| | QFNSTYRVVSVLTVLHQDWLNGKEYKCKVSNK | |
| | GLPSSIEKTISKAKGQPREPQVYTLPPSQEEMTKN | |
| | QVSLTCLVKGFYPSDIAVEWESNGQPENNYKTTP | |
| | PVLDSDGSFLLYSKLTVDKSRWQEGNVFSCSVM | |
| | HEALHNHYTQKSLSLSLGK | |
| CD3 LC | QTVVTQEPSLTVSPGGTVTLTCRSSTGAVTTSNY | 20 |
| | ANWVQQKPGQAPRGLIGGTNKRAPGTPARFSGS | |
| | LLGGKAALTLSGVQPEDEAEYYCALWYSNLWV | |
| | FGGGTKLTVLGQPKAAPSVTLFPPSSEELQANKA | |
| | TLVCLISDFYPGAVTVAWKADSSPVKAGVETTTP | |
| | SKQSNNKYAASSYLSLTPEQWKSHRSYSCQVTH | |
| | EGSTVEKTVAPTECS | |

Provided herein are approved products with at least one approved indication, wherein said approved product comprises talquetamab-tgvs.

Also provided herein are approved products comprising talquetamab-tgvs, wherein the approved product is approved for treating relapsed or refractory multiple myeloma in a patient.

Provided herein are approved products comprising talquetamab-tgvs, wherein such approved product improves anti-cancer cell activity as measured by overall response rate, duration of response (DOR), DOR rate, or time to first response, relative to a patient with relapsed or refractory multiple myeloma who is not receiving treatment with a bispecific antibody.

Provided herein are approved products comprising talquetamab-tgvs and is reference listed on the basis of data demonstrating an improvement in anti-cancer cell activity as measured by overall response rate, duration of response (DOR), DOR rate, or time to first response, relative to a patient with relapsed or refractory multiple myeloma who is not receiving treatment with a bispecific antibody.

Also provided herein are pharmaceutical products comprising talquetamab-tgvs in a single-dose vial, wherein the pharmaceutical product is packaged, and wherein the package includes a label that identifies the pharmaceutical product as indicated as monotherapy for the treatment of adult patients with relapsed or refractory multiple myeloma, who have received at least four prior lines of therapy, including a proteasome inhibitor, an immunomodulatory agent, and an anti-CD38 monoclonal antibody.

In some embodiments, the label includes a warning that cytokine release syndrome (CRS) and/or neurologic toxicity (e.g., ICANS) can occur in patients that receive the pharmaceutical product. In some embodiments, the label indicates that the pharmaceutical product is available only through a REMS program.

Methods of Treatment with Approved Products

Disclosed herein are methods for treating relapsed or refractory multiple myeloma in a patient, the methods comprising administering any of the herein disclosed approved products or any of the herein disclosed combinations to the patient in an amount and manner that is described in a drug product label for the approved product. In some embodiments, the approved product is talquetamab-tgvs.

It is to be understood that the method of treatment embodiments described herein encompass the use of an approved product or combinations for the manufacture of a medicament and the approved product or combinations for use in treatment. Thus, also described herein are uses of the approved product or combinations in the manufacture of a medicament for the treatment of multiple myeloma in a patient, wherein the medicament is administered in an amount that is described in the approved product label for the approved product.

Prior to the administering of the approved product, the methods can comprise administering a corticosteroid, an antihistamine, and an antipyretic to the patient. The methods for treating relapsed or refractory multiple myeloma in a patient can comprise administering a corticosteroid, an antihistamine, an antipyretic, and any of the herein disclosed approved products to the patient in an amount and manner that is described in a drug product label for the approved product. In some embodiments, the corticosteroid is dexamethasone, the antihistamine is diphenhydramine, and the antipyretic is acetaminophen. Thus, provided herein are methods for treating relapsed or refractory multiple myeloma in a patient, the methods comprising administering dexamethasone, diphenhydramine, acetaminophen, and any of the herein disclosed approved products to the patient in an amount and manner that is described in a drug product label for the approved product. The corticosteroid can comprise oral or intravenous dexamethasone, the antihistamine can comprise oral or intravenous diphenhydramine, and the antipyretic can comprise oral or intravenous acetaminophen. In some embodiments, the methods comprise administering 16 mg or equivalent of oral or intravenous dexamethasone, 50 mg or equivalent of oral or intravenous diphenhydramine, 650 mg to 1000 mg or equivalent of oral or intravenous acetaminophen, and any of the herein disclosed approved products to the patient in an amount and manner that is described in a drug product label for the approved product. In some embodiments, the corticosteroid, the antihistamine, and the antipyretic are administered to the patient prior to a step-up dose or a treatment dose. In some embodiments, the methods comprise administering 16 mg or equivalent of oral or intravenous dexamethasone, 50 mg or equivalent of oral or intravenous diphenhydramine, and 650 mg to 1000 mg or equivalent of oral or intravenous acetaminophen prior to each step-up dose and a treatment dose.

In some embodiments, the methods comprise reducing the patient's risk of CRS and/or neurologic toxicity (e.g., ICANS) by administering the approved product to the patient in accordance with a REMS program.

In some embodiments, the patient has not had T-cell redirection therapy within 3 months, prior Grade 3 or higher CRS related to any T-cell redirection therapy, an autologous stem cell transplant within the past 12 weeks, an allogenic stem cell transplant within the past 6 months, Eastern Cooperative Oncology Group (ECOG) performance score of 3 or higher, stroke or seizure within the past 6 months, CNS involvement or clinical signs of meningeal involvement of multiple myeloma, and plasma cell leukemia, active or documented history of autoimmune disease (exception of vitiligo, resolved childhood atopic dermatitis, resolved Grave's Disease that is euthyroid based on clinical and laboratory testing).

The administration of the approved product can provide improved anti-cancer cell activity as measured by overall response rate, duration of response (DOR), DOR rate, or time to first response, relative to a patient with multiple myeloma who is not receiving treatment with a bispecific antibody.

The administration of the approved product can provide improved anti-cancer cell activity as measured by overall response rate relative to a patient with multiple myeloma who is not receiving treatment with a bispecific antibody.

The administration of the approved product can provide improved anti-cancer cell activity as measured by stringent complete response (sCR) data relative to a patient with multiple myeloma who is not receiving treatment with a bispecific antibody.

The administration of the approved product can provide improved anti-cancer cell activity as measured by complete response (CR) data relative to a patient with multiple myeloma who is not receiving treatment with a bispecific antibody.

The administration of the approved product can provide improved anti-cancer cell activity as measured by very good partial response (VGPR) data relative to a patient with multiple myeloma who is not receiving treatment with a bispecific antibody.

The administration of the approved product can provide improved anti-cancer cell activity as measured by partial response (PR) data relative to a patient with multiple myeloma who is not receiving treatment with a bispecific antibody.

The administration of the approved product can provide improved anti-cancer cell activity as measured by stringent complete response data, complete response data, very good partial response data, and partial response data relative to a patient with multiple myeloma who is not receiving treatment with a bispecific antibody.

The administration of the approved product can provide improved anti-cancer cell activity in a patient population as measured by overall response rate relative to a patient population with multiple myeloma who are not receiving treatment with a bispecific antibody.

The administration of the approved product can provide improved anti-cancer cell activity in a patient population as measured by stringent complete response (sCR) data relative to a patient population with multiple myeloma who are not receiving treatment with a bispecific antibody.

The administration of the approved product can provide improved anti-cancer cell activity in a patient population as measured by complete response (CR) data relative to a patient population with multiple myeloma who are not receiving treatment with a bispecific antibody.

The administration of the approved product can provide improved anti-cancer cell activity in a patient population as measured by very good partial response (VGPR) data relative to a patient population with multiple myeloma who are not receiving treatment with a bispecific antibody.

The administration of the approved product can provide improved anti-cancer cell activity in a patient population as measured by partial response (PR) data relative to a patient population with multiple myeloma who are not receiving treatment with a bispecific antibody.

The administration of the approved product can provide improved anti-cancer cell activity in a patient population as measured by stringent complete response data, complete response data, very good partial response data, and partial response data relative to a patient population with multiple myeloma who are not receiving treatment with a bispecific antibody.

The administration of the approved product can provide improved anti-cancer cell activity as measured by duration of response (DOR) relative to a patient with multiple myeloma who is not receiving treatment with a bispecific antibody.

The administration of the approved product can provide improved anti-cancer cell activity in a patient population as measured by duration of response (DOR) relative to a patient population with multiple myeloma who are not receiving treatment with a bispecific antibody.

The administration of the approved product can provide improved anti-cancer cell activity as measured by time to first response relative to a patient with multiple myeloma who is not receiving treatment with a bispecific antibody.

The administration of the approved product can provide improved anti-cancer cell activity in a patient population as measured by time to first response relative to a patient population with multiple myeloma who are not receiving treatment with a bispecific antibody.

The administration of the approved product can provide improved anti-cancer cell activity as measured by duration of response rate relative to a patient with multiple myeloma who is not receiving treatment with a bispecific antibody.

The administration of the approved product can provide improved anti-cancer cell activity in a patient population as measured by duration of response rate relative to a patient population with multiple myeloma who are not receiving treatment with a bispecific antibody.

The improvement in anti-cancer cell activity can be relative to treatment with placebo.

The improvement in anti-cancer cell activity can be relative to no treatment.

The improvement in anti-cancer cell activity can be relative to standard of care.

The administration of an amount of the approved product can result in no more than a grade 2 adverse event.

The administration of an amount of the approved product can result in no more than a grade 3 adverse event.

The administration of an amount of the approved product can result in no more than a grade 4 adverse event.

### Methods of Restarting Therapy After a Dose Delay

Provided herein are methods of restarting a subject's dosing regimen for a GPRC5DxCD3 bispecific antibody after the subject has initiated a planned dosing regimen for treatment of multiple myeloma, and a delay has occurred between doses of the GPRC5DxCD3 bispecific antibody during the planned dosing regimen. According to certain embodiments, a planned dosing regimen comprises a step-up dosing schedule that includes a first step-up dose of 0.01 mg/kg, a second step-up dose of 0.06 mg/kg between 2 to 7 days after the first step-up dose, a first treatment dose of 0.4 mg/kg between 2 to 7 days after the second step-up dose; and subsequent treatment doses of 0.4 mg/kg one week after the first treatment dose and weekly thereafter. According to certain embodiments, a planned dosing regimen comprises a step-up dosing schedule that includes a first step-up dose of 0.01 mg/kg, a second step-up dose of 0.06 mg/kg between 2 to 7 days after the first step-up dose, a third step-up dose of 0.4 mg/kg between 2 to 7 days after the second step-up dose, a first treatment dose of 0.8 mg/kg between 2 to 7 days after the third step-up dose; and subsequent treatment doses of 0.8 mg/kg two weeks after the first treatment dose and every two weeks thereafter.

Dosing regimens of a pharmaceutical product are developed based on extensive pre-clinical and clinical data. Once a safe and effective dosing regimen is established for a given therapy (also referred to herein as a planned dosing regimen), subjects must adhere to the regimen or otherwise face significant risks, such as increased side effects and/or diminished efficacy, etc. However, it is not uncommon for subjects to experience dose delays, especially a subject with multiple myeloma that faces day-to-day health and lifestyle challenges. When subjects experience a delay between doses of a GPRC5DxCD3 bispecific antibody that is longer than the interval between those doses in the established (planned) dosing regimen, there is a need for methods of restarting the therapy that are safe and effective. The inventors have developed methods of restarting therapy on a GPRC5DxCD3 bispecific antibody that are tailored to the length and timing of a particular dose delay, i.e., a method is selected from several options based on the last dose amount administered to the subject and the number of days since the last dose administered. The methods enable subjects to receive a dosing regimen that is safe and effective in treating multiple myeloma, despite experiencing a delay in dosing.

According to certain embodiments, if a dose of the approved product is delayed, the patient restarts therapy based on Table A or Table B and resumes the weekly or biweekly (every 2 weeks) dosing schedule accordingly.

TABLE A
Recommendations for Restarting TALVEY after Dose Delay-Weekly
Dosing Schedule

| | Time from | |
|---|---|---|
| Last Dose | Last Dose | |
| Administered | Administered | TALVEY Recommendation* |
| 0.01 mg/kg | More than 7 | Restart TALVEY step-up dosing schedule at |
| | days | step-up dose 1 (0.01 mg/kg). |
| 0.06 mg/kg | 8 to 28 days | Repeat step-up dose 2 (0.06 mg/kg) and |
| | | continue TALVEY step-up dosing schedule. |
| | More than 28 | Restart TALVEY step-up dosing schedule at |
| | days | step-up dose 1 (0.01 mg/kg). |
| 0.4 mg/kg | 8 to 28 days | Continue TALVEY dosing schedule at |
| | | treatment dose (0.4 mg/kg weekly). |
| | 29 to 56 days | Restart TALVEY step-up dosing schedule at |
| | | step-up dose 2 (0.06 mg/kg). |
| | More than | Consider permanent discontinuation. If |
| | 56 days | restarting TALVEY, begin with the step-up |
| | | dosing schedule at step-up dose 1 (0.01 |
| | | mg/kg). |

*Administer pretreatment medications prior to restarting TALVEY. After restarting TALVEY, resume weekly dosing schedule accordingly [see Dosage and Administration (2.2) of the approved label].

TABLE B
Recommendations for Restarting TALVEY after Dose Delay-
Biweekly (Every 2 Weeks) Dosing Schedule

| Time from | |
|---|---|
| Last Dose | |
| Administered | TALVEY Recommendation* |
| More than 7 | Restart TALVEY step-up dosing schedule at step-up dose 1 |
| days | (0.01 mg/kg). |
| 8 to 28 days | Repeat step-up dose 2 (0.06 mg/kg) and continue TALVEY |
| | step-up dosing schedule. |
| More than 28 | Restart TALVEY step-up dosing schedule at step-up dose 1 |
| days | (0.01 mg/kg). |
| 8 to 28 days | Repeat step-up dose 3 (0.4 mg/kg) and continue TALVEY |
| | step-up dosing schedule. |
| 29 to 56 days | Restart TALVEY step-up dosing schedule at step-up dose 2 |
| | (0.06 mg/kg). |
| More than 56 | Consider permanent discontinuation. If restarting TALVEY, |
| days | begin with the step-up dosing schedule at step-up dose 1 |
| | (0.01 mg/kg). |
| 15 to 28 days | Continue TALVEY dosing schedule at treatment dose |
| | (0.8 mg/kg every 2 weeks). |
| 29 to 56 days | Restart TALVEY step-up dosing schedule at step-up |
| | dose 3 (0.4 mg/kg). |
| More than 56 | Consider permanent discontinuation. If restarting TALVEY, |
| days | begin with the step-up dosing schedule at step-up dose 1 |
| | (0.01 mg/kg). |

*Administer pretreatment medications prior to restarting TALVEY. After restarting TALVEY, resume biweekly (every 2 weeks) dosing schedule accordingly [see Dosage and Administration (2.2) of the approved label].

### Pharmaceutical Compositions and Routes of Administration

In view of its useful pharmacological properties, the approved products described herein may be formulated into various pharmaceutical forms for administration purposes.

The pharmaceutical compositions (e.g., formulations) can comprise any of the herein disclosed approved products and one or more pharmaceutically acceptable carriers, adjuvants, excipients, diluents, fillers, buffers, stabilizers, preservatives, lubricants, or other materials well known to those skilled in the art, and optionally other therapeutic or prophylactic agents. In some embodiments, the approved product is talquetamab-tgvs.

To prepare the pharmaceutical compositions, an effective amount of the approved product is combined in intimate admixture with a pharmaceutically acceptable carrier, which carrier may take a wide variety of forms depending on the form of preparation desired for administration. The pharmaceutical compositions can comprise an effective amount of the approved product combined in intimate admixture with one or more pharmaceutically acceptable excipients. Suitable pharmaceutically acceptable excipients include edetate disodium, glacial acetic acid, polysorbate 20, sodium acetate, sucrose, and water for injection, USP.

The pharmaceutical compositions can be in any form suitable for oral, intravenous, intraperitoneal, subcutaneous, pulmonary, transdermal, intramuscular, intranasal, buccal, sublingual, or suppository administration. The pharmaceutical compositions preferably are suitable for parenteral administration. The term “parenteral,” as used herein, includes intravenous, intramuscular, subcutaneous, rectal, vaginal, and intraperitoneal administration. More preferably, the pharmaceutical compositions are suitable for peripheral systemic delivery by intravenous, intraperitoneal, or subcutaneous injection. Most preferably, the pharmaceutical composition of the approved product is administered by subcutaneous injection.

These pharmaceutical compositions are desirably in unitary dosage form suitable, preferably, for parenteral injection. For example, the pharmaceutical compositions can be prepared by mixing the biological product included in the approved product with optional pharmaceutically acceptable carriers, excipients or stabilizers (Remington's Pharmaceutical Sciences 16th edition, Osol, A. Ed. (1980)), in the form of lyophilized formulations or aqueous solutions. The pharmaceutical composition comprising the approved product can comprise one or more excipients selected from edetate disodium, glacial acetic acid, polysorbate 20, sodium acetate, sucrose, and water.

Also provided herein are methods of preparing a pharmaceutical composition that comprises the approved product, in the form of an aqueous solution, characterized by combining the approved product with one or more excipients selected from edetate disodium, glacial acetic acid, polysorbate 20, sodium acetate, sucrose, and water for injection, USP. The aqueous solution can comprise talquetamab-tgvs, edetate disodium, glacial acetic acid, polysorbate 20, sodium acetate, sucrose, and water for injection, USP.

In some embodiments, the approved product is formulated to provide patients with a dose of about 0.01 mg/kg, 0.06 mg/kg, 0.4 mg/kg, or 0.8 mg/kg of talquetamab-tgvs in accordance with the approved product label.

In some embodiments, the dose of 0.01 mg/kg is administered using 2 mg/mL of talquetamab-tgvs in a single-dose vial, the dose of 0.06 mg/kg is administered using 2 mg/mL of talquetamab-tgvs in a single-dose vial, and each dose of 0.4 mg/kg and 0.8 mg/kg is administered using the 40 mg/mL of talquetamab-tgvs in a single-dose vial.

### Methods of Dosing and Treatment Regimens

The approved product is administered in an amount sufficient to exert its anti-cancer cell activity. The recommended dosing amounts, regimens, and other key elements of uses of the approved product is provided in Tables 1 and 2 and Example 1. In some embodiments, a patient is provided with a step-up dose 1 of the approved product in the amount of 0.01 mg/kg on day 1, followed by a step-up dose 2 of the approved product in the amount of 0.06 mg/kg on day 4, followed by a first treatment dose of the approved product in the amount of 0.4 mg/kg on day 7. The patient is then provided with a weekly dosing schedule of the approved product in the amount of 0.4 mg/kg that begins one week after the first treatment dose and is continued weekly thereafter. In some embodiments, a patient is provided with a step-up dose 1 of the approved product in the amount of 0.01 mg/kg on day 1, followed by a step-up dose 2 of the approved product in the amount of 0.06 mg/kg on day 4, followed by a step-up dose 3 of the approved product in the amount of 0.4 mg/kg on day 7, followed by a first treatment dose of the approved product in the amount of 0.8 mg/kg on day 10. The patient is then provided with a biweekly (every 2 weeks) dosing schedule of the approved product in the amount of 0.8 mg/kg that begins two weeks after the first treatment dose and is continued biweekly thereafter. There are other variations on dosing that are also described in Example 1. In some embodiments, the approved product is talquetamab-tgvs.

The patient is a patient who has received at least four prior lines of therapy for the treatment of multiple myeloma including a proteasome inhibitor, an immunomodulatory agent, and an anti-CD38 monoclonal antibody.

The patient is pre-treated with one or more secondary medicinal products prior to receiving the approved product. A patient may be administered one or more pre-treatment medicinal products about 10 minutes to 6 hours, more specifically 30 minutes to 4.5 hours, or more specifically 1 hour to 3 hours prior to administration of the approved product. The one or more pre-treatment medicinal products include a corticosteroid, antihistamine, and/or an antipyretic. In an embodiment, the corticosteroid is oral or intravenous dexamethasone. In an embodiment, the antihistamine is oral or intravenous diphenhydramine. In an embodiment, the antipyretic is oral or intravenous acetaminophen. In an embodiment, the patient is administered about 16 mg or equivalent of oral or intravenous dexamethasone, about 50 mg or equivalent of oral or intravenous diphenhydramine, and about 650 mg to 1000 mg or equivalent of oral or intravenous acetaminophen one to three hours prior to administration of a step-up dose or a treatment dose of the approved product. Additional details regarding pre-treatment are provided in Example 1.

Also provided herein are methods of reducing the risk of an adverse event in a patient that receives an approved product. In an embodiment, the method comprises reducing the risk of cytokine release syndrome (CRS) and/or neurologic toxicity, such as Immune Effector Cell-Associated Neurotoxicity Syndrome (ICANS), in a patient that receives the approved product. In an embodiment, the method comprises administering the approved product in accordance with a Risk Evaluation and Mitigation Strategy (REMS) program (e.g., as described at www.TEC-TALREMS, the contents of which are incorporated by reference herein). As used herein, a REMS program is a program required by a health authority to manage known or potential serious risks associated with an approved product to ensure the benefits of the approved product outweigh its risks. According to an embodiment, a method for treating relapsed or refractory multiple myeloma in a patient comprises administering the approved product in accordance with a REMS program.

According to an embodiment, the REMS program includes distributing educational materials to healthcare providers (HCPs) and pharmacies that describe risks of CRS and/or neurologic toxicity associated with the approved product. The REMS program may include Elements to Assure Safe Use (ETASU), which comprises requiring prescribers to complete a training program on the risks of CRS and/or neurologic toxicity associated with the approved product, in order to become certified to prescribe the approved product. According to an embodiment, the REMS program comprises requiring prescribers to counsel patients on how to recognize and respond to signs and symptoms of CRS and neurologic toxicity (e.g., ICANS); for example, the REMS program may include training prescribers to instruct a patient that the patient should be hospitalized for 48 hours after administration of all doses within the step-up dosing schedule for the approved product. According to an embodiment, the REMS program comprises requiring pharmacies and healthcare settings to become certified to dispense the approved product by completing a training program.

According to an embodiment, administering an approved product to a patient in accordance with a REMS program comprises, prior to administering the approved product to the patient: (1) becoming certified to prescribe the approved product by completing a training program about risks of CRS and/or neurologic toxicity associated with the approved product; and (2) counseling the patient on how to recognize and respond to signs and symptoms of CRS and neurologic toxicity (e.g., ICANS).

### Approved Products as Kits or Articles of Manufacture

Also provided herein are kits and articles of manufacture comprising any of the herein disclosed approved products, any of the herein disclosed combinations, or any of the herein disclosed pharmaceutical products (“kits” shall be construed to include the elements of the approved product as defined herein). Such kits include a package or container that is compartmentalized to receive one or more dosages of the approved product disclosed herein. Suitable containers include, for example, bottles or vials. The containers can be formed from a variety of materials such as glass or plastic.

In some embodiments, the approved product is talquetamab-tgvs.

The articles of manufacture provided herein contain packaging materials. Examples of pharmaceutical packaging materials include, but are not limited to, blister packs, bottles, tubes, bags, containers, syringes, and any packaging material suitable for a selected formulation and intended mode of administration and treatment.

A kit typically includes labels listing contents and/or instructions for use, and package inserts with instructions for use. A set of instructions will also typically be included.

A label is on or associated with the container. In an embodiment, a label is on a container when letters, numbers or other characters forming the label are attached, molded or etched into the container itself; a label is associated with a container when it is present within a receptacle or carrier that also holds the container, e.g., as a package insert. An example of a kit is a kit comprising TALVEY™. A further example is a kit comprising a biosimilar. An example of an article of manufacture is an article of manufacture comprising TALVEY™. A further example is an article of manufacture comprising a biosimilar.

A label is used to indicate that the contents of a kit or of an article of manufacture are to be used for a specific therapeutic application. The label also indicates directions for use of the contents, such as in the methods described herein. The kit or the article of manufacture can comprise the approved product label.

The kits may comprise one or more suitably aliquoted compositions or reagents to generate compositions of the disclosure. The components of the kits may be packaged either in aqueous media or in lyophilized form. The container means of the kits may include at least one vial, test tube, flask, bottle, syringe, or other container means, into which a component may be placed, and preferably, suitably aliquoted. Where there is more than one component in the kit, the kit also will generally contain a second, third, or other additional containers into which the additional components may be separately placed. However, various combinations of components may be comprised in a vial. The kits also will typically include a means for containing the approved product and any other reagent containers in close confinement for commercial sale. Such containers may include injection or blow molded plastic containers into which the desired vials are retained, for example.

In one embodiment, the vial or dispenser device is accompanied by instructions for administration. In one embodiment, the vial or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Methods of Negotiating a Maximum Fair Price, Methods of Selling, and Methods of Offering For Sale an Approved Product

Provided herein are methods for negotiating a maximum fair price for an approved product that is negotiation eligible, for example, in accordance with H.R. 5376 (Inflation Reduction Act of 2022), which is incorporated by reference herein. An embodiment of the methods comprise identifying the approved product as negotiation eligible, selecting the negotiation eligible approved product to be included in a price negotiation with a license holder of the approved product, wherein the license holder and a third party enter into negotiations to determine a maximum fair price for the negotiation eligible approved product, and concluding the negotiations with an agreed-upon maximum fair price for the negotiation eligible approved product. As used herein, the term “maximum fair price” means, with respect to a plan year during a price applicability period and with respect to a selected drug (as defined in section 1192(c)) with respect to such period, the price published pursuant to section 1195 in the Federal Register for such drug and year.

Also provided herein are methods of selling an approved product, the methods comprising selling the approved product, wherein an approved product label for a reference product for the approved product includes instructions for treating adult patients with relapsed or refractory multiple myeloma after at least four prior lines of therapy, including a proteasome inhibitor, an immunomodulatory agent, and an anti-CD38 monoclonal antibody. In some embodiments, the methods comprise selling talquetamab-tgvs.

In some embodiments, the methods of selling comprise selling a biosimilar, wherein an approved product label for a reference product for the biosimilar includes instructions for treating adult patients with relapsed or refractory multiple myeloma after at least four prior lines of therapy, including a proteasome inhibitor, an immunomodulatory agent, and an anti-CD38 monoclonal antibody.

Also provided herein are methods of offering for sale an approved product, the methods comprising offering for sale the approved product, wherein an approved product label for a reference product for the approved product includes instructions for treating adult patients with relapsed or refractory multiple myeloma after at least four prior lines of therapy, including a proteasome inhibitor, an immunomodulatory agent, and an anti-CD38 monoclonal antibody. In some embodiments, the methods comprise offering talquetamab-tgvs for sale.

In some embodiments, the methods of offering for sale comprise offering for sale a biosimilar, wherein an approved product label for a reference product for the biosimilar includes instructions for treating adult patients with relapsed or refractory multiple myeloma after at least four prior lines of therapy, including a proteasome inhibitor, an immunomodulatory agent, and an anti-CD38 monoclonal antibody. In some embodiments, the biosimilar is a biosimilar of talquetamab-tgvs.

Provided herein are methods of selling of kits. The method comprising selling the kits, wherein an approved product label for a reference product for the approved product includes instructions for treating multiple myeloma such as those described in Example 1. In some embodiments, the method comprises selling the kit, wherein an approved product label for a reference product for the approved product includes instructions for treating a patient with relapsed or refractory multiple myeloma after at least four prior lines of therapy, including a proteasome inhibitor, an immunomodulatory agent, and an anti-CD38 monoclonal antibody.

In some embodiments, the methods of selling comprise methods of selling a kit comprising a biosimilar, wherein an approved product label for a reference product for the biosimilar includes instructions for treating a patient with relapsed or refractory multiple myeloma, and in some embodiments, for treating a patient with relapsed or refractory multiple myeloma after at least four prior lines of therapy, including a proteasome inhibitor, an immunomodulatory agent, and an anti-CD38 monoclonal antibody.

The disclosure further provides methods of offering for sale a kit comprising talquetamab-tgvs, the methods comprising offering for sale the kit, wherein the approved product label for a reference product for talquetamab-tgvs includes instructions for treating a patient with relapsed or refractory multiple myeloma, and in some embodiments, for treating a patient with relapsed or refractory multiple myeloma after at least four prior lines of therapy, including a proteasome inhibitor, an immunomodulatory agent, and an anti-CD38 monoclonal antibody.

Also described herein are methods of selling a kit comprising a biosimilar or the approved product, wherein the approved product label for the biosimilar or the approved product comprises overall response rate data, duration of response data, duration of response rate, and/or time to first response. In some embodiments, the biosimilar label that is similar to or the same as the approved product label comprises overall response rate data. In some embodiments, the overall response rate data is stringent complete response data. In some embodiments, the overall response rate data is complete response data. In some embodiments, the overall response rate data is very good partial response data. In some embodiments, the overall response rate data is partial response data. An example of the overall response rate data is stringent complete response data, complete response data, very good partial response data, and partial response data.

In some embodiments, the overall response rate data for a biosimilar or the approved product is at least 70% in patients receiving 0.4 mg/kg weekly or 0.8 mg/kg biweekly, in particular, wherein the patients have relapsed or refractory multiple myeloma. In some embodiments, the overall response rate data for a biosimilar or the approved product is at least 70% in patients receiving 0.4 mg/kg weekly or 0.8 mg/kg biweekly, in particular, wherein the patients have relapsed or refractory multiple myeloma, who have received at least three prior lines of therapy, including a proteasome inhibitor, immunomodulatory agent, and anti-CD38 monoclonal antibody.

In some embodiments, the overall response rate data for a biosimilar or the approved product is at least 71% in patients receiving 0.4 mg/kg weekly or 0.8 mg/kg biweekly, in particular, wherein the patients have relapsed or refractory multiple myeloma. In some embodiments, the overall response rate data for a biosimilar or the approved product is at least 71% in patients receiving 0.4 mg/kg weekly or 0.8 mg/kg biweekly, in particular, wherein the patients have relapsed or refractory multiple myeloma, who have received at least three prior lines of therapy, including a proteasome inhibitor, immunomodulatory agent, and anti-CD38 monoclonal antibody.

In some embodiments, the overall response rate data for a biosimilar or the approved product is at least 73% in patients receiving 0.4 mg/kg weekly or at least 73.6% in patients receiving 0.8 mg/kg biweekly, in particular, wherein the patients have relapsed or refractory multiple myeloma. In some embodiments, the overall response rate data for a biosimilar or the approved product is at least 73% in patients receiving 0.4 mg/kg weekly or at least 73.6% in patients receiving 0.8 mg/kg biweekly, in particular, wherein the patients have relapsed or refractory multiple myeloma, who have received at least three prior lines of therapy, including a proteasome inhibitor, immunomodulatory agent, and anti-CD38 monoclonal antibody.

In some embodiments, the stringent complete response (sCR) data for a biosimilar or the approved product is at least 26% in patients receiving 0.4 mg/kg weekly or at least 20% in patients receiving 0.8 mg/kg biweekly, in particular, wherein the patients have relapsed or refractory multiple myeloma, who have received at least three prior lines of therapy, including a proteasome inhibitor, immunomodulatory agent, and anti-CD38 monoclonal antibody.

In some embodiments, the complete response (CR) data for a biosimilar or the approved product is at least 9% in patients receiving 0.4 mg/kg weekly or at least 13% in patients receiving 0.8 mg/kg biweekly, in particular, wherein the patients have relapsed or refractory multiple myeloma, who have received at least three prior lines of therapy, including a proteasome inhibitor, immunomodulatory agent, and anti-CD38 monoclonal antibody.

In some embodiments, the very good partial response (VGPR) data for a biosimilar or the approved product is at least 22% in patients receiving 0.4 mg/kg weekly or at least 25% in patients receiving 0.8 mg/kg biweekly, in particular, wherein the patients have relapsed or refractory multiple myeloma, who have received at least three prior lines of therapy, including a proteasome inhibitor, immunomodulatory agent, and anti-CD38 monoclonal antibody.

In some embodiments, the partial response (PR) data for a biosimilar or the approved product is at least 16% in patients receiving 0.4 mg/kg weekly or at least 16% in patients receiving 0.8 mg/kg biweekly, in particular, wherein the patients have relapsed or refractory multiple myeloma, who have received at least three prior lines of therapy, including a proteasome inhibitor, immunomodulatory agent, and anti-CD38 monoclonal antibody.

In some embodiments, the duration of response (DOR) data for a biosimilar or the approved product is at least 9.5 months in patients receiving 0.4 mg/kg weekly, in particular, wherein the patients have relapsed or refractory multiple myeloma, who have received at least three prior lines of therapy, including a proteasome inhibitor, immunomodulatory agent, and anti-CD38 monoclonal antibody.

In some embodiments, the median time to first response data for a biosimilar or the approved product is at least 1.2 months in patients receiving 0.4 mg/kg weekly or at least 1.3 months in patients receiving 0.8 mg/kg biweekly, in particular, wherein the patients have relapsed or refractory multiple myeloma, who have received at least three prior lines of therapy, including a proteasome inhibitor, immunomodulatory agent, and anti-CD38 monoclonal antibody.

Still further provided herein are methods of improving overall response rate, duration of response, and time to first response in patients with relapsed or refractory multiple myeloma, the methods comprising administering to the patients a biosimilar or the approved product. Also provided herein are methods of improving overall response rate data in patients with relapsed or refractory multiple myeloma, the methods comprising administering to the patients a biosimilar or the approved product. Also provided herein are methods of improving stringent complete response data in patients with relapsed or refractory multiple myeloma, the methods comprising administering to the patients a biosimilar or the approved product. Also provided herein are methods of improving complete response data in patients with relapsed or refractory multiple myeloma, the methods comprising administering to the patients a biosimilar or the approved product. Also provided herein are methods of improving very good partial response data in patients with relapsed or refractory multiple myeloma, the methods comprising administering to the patients a biosimilar or the approved product. Also provided herein are methods of improving partial response data in patients with relapsed or refractory multiple myeloma, the methods comprising administering to the patients a biosimilar or the approved product. Also provided herein are methods of improving duration of response data in patients with relapsed or refractory multiple myeloma, the methods comprising administering to the patients a biosimilar or the approved product. Also provided herein are methods of improving time to first response data in patients with relapsed or refractory multiple myeloma, the methods comprising administering to the patients a biosimilar or the approved product. Also provided herein are methods of improving duration of response rate data in patients with relapsed or refractory multiple myeloma, the methods comprising administering to the patients a biosimilar or the approved product.

In some embodiments, the overall response rate data for a biosimilar or the approved product is at least 70% in patients receiving 0.4 mg/kg weekly or 0.8 mg/kg biweekly, in particular, wherein the patients have relapsed or refractory multiple myeloma. In some embodiments, the overall response rate data for a biosimilar or the approved product is at least 73% in patients receiving 0.4 mg/kg weekly or at least 73.6% in patients receiving 0.8 mg/kg biweekly, in particular, wherein the patients have relapsed or refractory multiple myeloma. In some embodiments, the overall response rate data for a biosimilar or the approved product is at least 73% in patients receiving 0.4 mg/kg weekly or at least 73.6% in patients receiving 0.8 mg/kg biweekly, in particular, wherein the patients have relapsed or refractory multiple myeloma, who have received at least three prior lines of therapy, including a proteasome inhibitor, immunomodulatory agent, and anti-CD38 monoclonal antibody. In some embodiments, the stringent complete response (sCR) data for a biosimilar or the approved product is at least 26% in patients receiving 0.4 mg/kg weekly or at least 20% in patients receiving 0.8 mg/kg biweekly, in particular, wherein the patients have relapsed or refractory multiple myeloma, who have received at least three prior lines of therapy, including a proteasome inhibitor, immunomodulatory agent, and anti-CD38 monoclonal antibody. In some embodiments, the complete response (CR) data for a biosimilar or the approved product is at least 9% in patients receiving 0.4 mg/kg weekly or at least 13% in patients receiving 0.8 mg/kg biweekly, in particular, wherein the patients have relapsed or refractory multiple myeloma, who have received at least three prior lines of therapy, including a proteasome inhibitor, immunomodulatory agent, and anti-CD38 monoclonal antibody. In some embodiments, the very good partial response (VGPR) data for a biosimilar or the approved product is at least 22% in patients receiving 0.4 mg/kg weekly or at least 25% in patients receiving 0.8 mg/kg biweekly, in particular, wherein the patients have relapsed or refractory multiple myeloma, who have received at least three prior lines of therapy, including a proteasome inhibitor, immunomodulatory agent, and anti-CD38 monoclonal antibody. In some embodiments, the partial response (PR) data for a biosimilar or the approved product is at least 16% in patients receiving 0.4 mg/kg weekly or at least 16% in patients receiving 0.8 mg/kg biweekly, in particular, wherein the patients have relapsed or refractory multiple myeloma, who have received at least three prior lines of therapy, including a proteasome inhibitor, immunomodulatory agent, and anti-CD38 monoclonal antibody. In some embodiments, the duration of response (DOR) data for a biosimilar or the approved product is at least 9.5 months in patients receiving 0.4 mg/kg weekly, in particular, wherein the patients have relapsed or refractory multiple myeloma, who have received at least three prior lines of therapy, including a proteasome inhibitor, immunomodulatory agent, and anti-CD38 monoclonal antibody. In some embodiments, the median time to first response data for a biosimilar or the approved product is at least 1.2 months in patients receiving 0.4 mg/kg weekly or at least 1.3 months in patients receiving 0.8 mg/kg biweekly, in particular, wherein the patients have relapsed or refractory multiple myeloma, who have received at least three prior lines of therapy, including a proteasome inhibitor, immunomodulatory agent, and anti-CD38 monoclonal antibody.

### ENUMERATED EMBODIMENTS

Provided below are enumerated embodiments. These embodiments are illustrative only and do not limit the scope of the present disclosure or of the claims attached hereto.

1. An approved product comprising:
3 mg/1.5 mL (2 mg/mL) of talquetamab-tgvs in a single-dose vial; and/or
40 mg/mL of talquetamab-tgvs in a single-dose vial.
   2. The approved product of embodiment 1, comprising 3 mg/1.5 mL (2 mg/mL) of talquetamab-tgvs in a single-dose vial.
   3. The approved product of embodiment 1, comprising 40 mg/mL of talquetamab-tgvs in a single-dose vial.
   4. The approved product of any one of the previous embodiments, wherein the approved product is formulated for delivery to a human in a dosing schedule comprising:
0.01 mg/kg administered as a first step-up dose on Day 1;
0.06 mg/kg administered as a second step-up dose 2 to 7 days after the first step-up dose;
0.4 mg/kg administered as a first treatment dose 2 to 7 days after the second step-up dose; and
0.4 mg/kg administered as a subsequent treatment dose one week after the first treatment dose and once weekly thereafter.
   5. The approved product of embodiment 4, wherein the second step-up dose is administered on Day 4.
   6. The approved product of embodiment 4 or 5, wherein the first treatment dose is administered on Day 7.
   7. The approved product of any one of the previous embodiments, wherein the approved product is formulated for delivery to a human in a dosing schedule comprising:
0.01 mg/kg administered as a first step-up dose on Day 1;
0.06 mg/kg administered as a second step-up dose on Day 4;
0.4 mg/kg administered as a first treatment dose on Day 7; and
0.4 mg/kg administered as a subsequent treatment dose one week after the first treatment dose and once weekly thereafter.
   8. The approved product of any one of embodiments 1-3, wherein the approved product is formulated for delivery to a human in a dosing schedule comprising:
0.01 mg/kg administered as a first step-up dose on Day 1;
0.06 mg/kg administered as a second step-up dose 2 to 7 days after the first step-up dose;
0.4 mg/kg administered as a third step-up dose 2 to 7 days after the second step-up dose;
0.8 mg/kg administered as a first treatment dose 2 to 7 days after the third step-up dose; and
0.8 mg/kg administered as a subsequent treatment dose 12 to 14 days after the first treatment dose and once every two weeks thereafter.
   9. The approved product of embodiment 8, wherein the second step-up dose is administered on Day 4.
   10. The approved product of embodiment 8 or 9, wherein the third step-up dose is administered on Day 7.
   11. The approved product of any one of embodiments 8-10, wherein the first treatment dose is administered on Day 10.
   12. The approved product of any one of embodiments 8-11, wherein the approved product is formulated for delivery to a human in a dosing schedule comprising:
0.01 mg/kg administered as a first step-up dose on Day 1;
0.06 mg/kg administered as a second step-up dose on Day 4;
0.4 mg/kg administered as a third step-up dose on Day 7;
0.8 mg/kg administered as a first treatment dose on Day 10; and
0.8 mg/kg administered as a subsequent treatment dose 12 to 14 days after the first treatment dose and once every two weeks thereafter.
   13. The approved product of any one of the previous embodiments, wherein the approved product is a biosimilar.
   14. The approved product of any one of the previous embodiments, in a 1.5 ml single-dose vial comprising 3 mg of talquetamab-tgvs, edetate disodium, glacial acetic acid, polysorbate 20, sodium acetate, sucrose, and water for injection, USP.
   15. The approved product of embodiment 14, in a 1.5 ml single-dose vial comprising 3 mg of talquetamab-tgvs, 0.027 mg edetate disodium, 0.36 mg glacial acetic acid, 0.6 mg polysorbate 20, 1.39 mg sodium acetate, 120 mg sucrose, and Water for Injection, USP.
   16. The approved product of any one of embodiments 1-13, in a 1.0 ml single-dose vial comprising 40 mg of talquetamab-tgvs, edetate disodium, glacial acetic acid, polysorbate 20, sodium acetate, sucrose, and water for injection, USP.
   17. The approved product of embodiment 16, in a 1.0 ml single-dose vial comprising 40 mg of talquetamab-tgvs, 0.018 mg edetate disodium, 0.24 mg glacial acetic acid, 0.4 mg polysorbate 20, 0.90 mg sodium acetate, 80 mg sucrose, and Water for Injection, USP.
   18. The approved product of any one of the previous embodiments, in combination with a corticosteroid, an antihistamine, and an antipyretic.
   19. The approved product of embodiment 18, in combination with 16 mg or equivalent of oral or intravenous dexamethasone, 50 mg or equivalent of oral or intravenous diphenhydramine, and 650 mg to 1000 mg, or equivalent, oral or intravenous acetaminophen.
   20. The approved product of any one of the previous embodiments, wherein, following administration and a median duration of follow-up of 13.8 months from first response among responders in a subject population, the median duration of response is at least 9 months in said subject population.
   21. The approved product of any one of the previous embodiments, wherein the approved product is administered to a subject in accordance with a REMS program to reduce a risk that the subject experiences cytokine release syndrome (CRS) and/or neurologic toxicity (e.g., ICANS) after receiving the approved product.
   22. A combination of a corticosteroid, an antihistamine, an antipyretic, and an approved product.
   23. The combination of embodiment 22, wherein the corticosteroid comprises oral or intravenous dexamethasone or equivalent, the antihistamine comprises oral or intravenous diphenhydramine or equivalent, and the antipyretic comprises oral or intravenous acetaminophen or equivalent.
   24. The combination of embodiment 23, wherein the combination comprises 16 mg or equivalent of oral or intravenous dexamethasone, 50 mg or equivalent of oral or intravenous diphenhydramine, and 650 mg to 1000 mg, or equivalent, oral or intravenous acetaminophen.
   25. A pharmaceutical product comprising talquetamab-tgvs in a single-dose vial, wherein the pharmaceutical product is packaged, and wherein the package includes a label that identifies the pharmaceutical product as indicated as monotherapy for the treatment of adult subjects with relapsed or refractory multiple myeloma, who have received at least four prior lines of therapy, including a proteasome inhibitor, an immunomodulatory agent, and an anti-CD38 monoclonal antibody.
   26. The pharmaceutical product of embodiment 25, wherein the label includes a warning that cytokine release syndrome (CRS) and/or neurologic toxicity (e.g., ICANS) can occur in subjects that receive the pharmaceutical product.
   27. The pharmaceutical product of embodiment 25 or 26, wherein the label indicates that the pharmaceutical product is available only through a REMS program.
   28. A kit comprising the approved product of any one of embodiments 1-21, the combination of any one of embodiments 22-24, or the pharmaceutical product of any one of embodiments 25-27.
   29. A method for treating relapsed or refractory multiple myeloma in a subject, the method comprising administering the approved product of any one of embodiments 1-21 or the combination of any one of embodiments 22-24 to the subject in an amount and manner that is described in a drug product label for the approved product.
   30. The method of embodiment 29, further comprising, prior to the administering of the approved product, administering a corticosteroid, an antihistamine, and an antipyretic to the subject.
   31. The method of embodiment 30, wherein the corticosteroid is dexamethasone, the antihistamine is diphenhydramine, and the antipyretic is acetaminophen.
   32. The method of embodiment 31, comprising administering 16 mg of oral or intravenous dexamethasone or equivalent, 50 mg of oral or intravenous diphenhydramine or equivalent, and 650 mg to 1000 mg of oral or intravenous acetaminophen or equivalent prior to the administering of the approved product.
   33. The method of any one of embodiments 29-32, wherein the corticosteroid, the antihistamine, and the antipyretic are administered to the subject prior to a step-up dose or a treatment dose.
   34. A method for treating relapsed or refractory multiple myeloma in a subject, the method comprising administering a corticosteroid, an antihistamine, an antipyretic, and the approved product of any one of embodiments 1-21 to the subject in an amount and manner that is described in a drug product label for the approved product.
   35. The method of embodiment 34, comprising administering 16 mg or equivalent of oral or intravenous dexamethasone, 50 mg or equivalent of oral or intravenous diphenhydramine, and 650 mg to 1000 mg, or equivalent, oral or intravenous acetaminophen prior to administering the approved product.
   36. The method of any one of embodiments 29-35, comprising reducing the subject's risk of CRS and/or neurologic toxicity (e.g., ICANS) by administering the approved product to the subject in accordance with a REMS program.
   37. A method of selling an approved product, the method comprising selling the approved product, wherein an approved product label for a reference product for the approved product includes instructions for treating adult subjects with relapsed or refractory multiple myeloma after at least four prior lines of therapy, including a proteasome inhibitor, an immunomodulatory agent, and an anti-CD38 monoclonal antibody.
   38. A method of offering for sale an approved product, the method comprising offering for sale the approved product, wherein an approved product label for a reference product for the approved product includes instructions for treating adult subjects with relapsed or refractory multiple myeloma after at least four prior lines of therapy, including a proteasome inhibitor, an immunomodulatory agent, and an anti-CD38 monoclonal antibody.
   39. A method of negotiating a maximum fair price for an approved product, the method comprising identifying the approved product as negotiation eligible, selecting the negotiation eligible approved product to be included in a price negotiation with a license holder of the approved product, wherein the license holder and a third party enter into negotiations to determine a maximum fair price for the negotiation eligible approved product, and concluding the negotiations with an agreed-upon maximum fair price for the negotiation eligible approved product.

Provided below are additional enumerated embodiments. These embodiments are illustrative only and do not limit the scope of the present disclosure or of the claims attached hereto.

1A. A method of restarting a subject's dosing regimen for a GPRC5DxCD3 bispecific antibody after the subject has initiated a planned dosing regimen for treatment of multiple myeloma, and a delay has occurred between doses of the GPRC5DxCD3 bispecific antibody during the planned dosing regimen, wherein the planned dosing regimen comprises:
(1) a step-up dosing schedule that includes (a) a first step-up dose of 0.01 mg/kg, (b) a second step-up dose of 0.06 mg/kg between 2 to 7 days after the first step-up dose, and (c) a first treatment dose of 0.4 mg/kg between 2 to 7 days after the second step-up dose; and
(2) a weekly dosing schedule that includes subsequent treatment doses of 0.4 mg/kg one week after the first treatment dose and weekly thereafter, with a minimum of 6 days between said treatment doses,
wherein the method of restarting the subject's dosing regimen comprises:
(i) if the last dose administered to the subject was 0.01 mg/kg, and the time from the last dose administered is more than 7 days, then restarting the step-up dosing schedule at the first step-up dose of 0.01 mg/kg and resuming the planned dosing regimen, or
(ii) if the last dose administered to the subject was 0.06 mg/kg, and the time from the last dose administered is 8 to 28 days, then repeating the second step-up dose of 0.06 mg/kg and continuing the step-up dosing schedule (thereby resuming the planned dosing regimen), or
(iii) if the last dose administered to the subject was 0.06 mg/kg, and the time from the last dose administered is more than 28 days, then restarting the step-up dosing schedule at the first step-up dose of 0.01 mg/kg and resuming the planned dosing regimen, or
(iv) if the last dose administered to the subject was 0.4 mg/kg, and the time from the last dose administered is 8 to 28 days, then continuing the planned dosing regimen at treatment doses of 0.4 mg/kg weekly (thereby resuming the planned dosing regimen), or
(v) if the last dose administered to the subject was 0.4 mg/kg, and the time from the last dose administered is 29 to 56 days, then restarting the step-up dosing schedule at the second step-up dose of 0.06 mg/kg and resuming the planned dosing regimen, or
(vi) if the last dose administered to the subject was 0.4 mg/kg, and the time from the last dose administered is more than 56 days, then either discontinuing the planned dosing regimen permanently, or restarting the step-up dosing schedule at the first step-up dose of 0.01 mg/kg and resuming the planned dosing regimen.
   2A. The method of embodiment TA, wherein the step-up dosing schedule includes (a) the first step-up dose of 0.01 mg/kg on Day 1, (b) the second step-up dose of 0.06 mg/kg on Day 4, and (c) the first treatment dose of 0.4 mg/kg on Day 7.
   3A. The method of embodiment TA, wherein the step-up dosing schedule includes (a) the first step-up dose of 0.01 mg/kg on Day 1, (b) the second step-up dose of 0.06 mg/kg on Day 3, and (c) the first treatment dose of 0.4 mg/kg on Day 5.
   4A. The method of any of embodiments 1A-3A, wherein each of the second step-up dose and the first treatment dose is administered between 2 to 4 days after the previous dose, or is optionally administered up to 7 days after the previous dose to allow for resolution of adverse reactions.
   5A. A method of restarting a subject's dosing regimen for a GPRC5DxCD3 bispecific antibody after the subject has initiated a planned dosing regimen for treatment of multiple myeloma, and a delay has occurred between doses of the GPRC5DxCD3 bispecific antibody during the planned dosing regimen, wherein the planned dosing regimen comprises:
(1) a step-up dosing schedule that includes (a) a first step-up dose of 0.01 mg/kg, (b) a second step-up dose of 0.06 mg/kg between 2 to 7 days after the first step-up dose, (c) a third step-up dose of 0.4 mg/kg between 2 to 7 days after the second step-up dose, and (d) a first treatment dose of 0.8 mg/kg between 2 to 7 days after the third step-up dose; and
(2) a biweekly (every two weeks) dosing schedule that includes subsequent treatment doses of 0.8 mg/kg two weeks after the first treatment dose and every two weeks thereafter, with a minimum of 12 days between said treatment doses,
wherein the method of restarting the subject's dosing regimen comprises:
(i) if the last dose administered to the subject was 0.01 mg/kg, and the time from the last dose administered is more than 7 days, then restarting the step-up dosing schedule at the first step-up dose of 0.01 mg/kg and resuming the planned dosing regimen, or
(ii) if the last dose administered to the subject was 0.06 mg/kg, and the time from the last dose administered is 8 to 28 days, then repeating the second step-up dose of 0.06 mg/kg, continuing the step-up dosing schedule (thereby resuming the planned dosing regimen), or
(iii) if the last dose administered to the subject was 0.06 mg/kg, and the time from the last dose administered is more than 28 days, then restarting the step-up dosing schedule at the first step-up dose of 0.01 mg/kg and resuming the planned dosing regimen, or
(iv) if the last dose administered to the subject was 0.4 mg/kg, and the time from the last dose administered is 8 to 28 days, then repeating the third step-up dose of 0.4 mg/kg and continuing the step-up dosing schedule (thereby resuming the planned dosing regimen), or
(v) if the last dose administered to the subject was 0.4 mg/kg, and the time from the last dose administered is 29 to 56 days, then restarting the step-up dosing schedule at the second step-up dose of 0.06 mg/kg and resuming the planned dosing regimen, or
(vi) if the last dose administered to the subject was 0.4 mg/kg, and the time from the last dose administered is more than 56 days, then either discontinuing the planned dosing regimen permanently, or restarting the step-up dosing schedule at the first step-up dose of 0.01 mg/kg and resuming the planned dosing regimen, or
(vii) if the last dose administered to the subject was 0.8 mg/kg, and the time from the last dose administered is 15 to 28 days, then continuing the planned dosing regimen at treatment doses of 0.8 mg/kg every two weeks (thereby resuming the planned dosing regimen); or
(viii) if the last dose administered to the subject was 0.8 mg/kg, and the time from the last dose administered is 29 to 56 days, then restarting the step-up dosing schedule at the third step-up dose of 0.4 mg/kg and resuming the planned dosing regimen, or
(ix) if the last dose administered to the subject was 0.8 mg/kg, and the time from the last dose administered is more than 56 days, then either discontinuing the planned dosing regimen permanently, or restarting the step-up dosing schedule at the first step-up dose of 0.01 mg/kg and resuming the planned dosing regimen.
   6A. The method of embodiment 5A, wherein the step-up dosing schedule includes (a) the first step-up dose of 0.01 mg/kg on Day 1, (b) the second step-up dose of 0.06 mg/kg on Day 4, (c) the third step-up dose of 0.4 mg/kg on Day 7, and (d) the first treatment dose of 0.8 mg/kg on Day 10.
   7A. The method of embodiment 5A, wherein the step-up dosing schedule includes (a) the first step-up dose of 0.01 mg/kg on Day 1, (b) the second step-up dose of 0.06 mg/kg on Day 3, (c) the third step-up dose of 0.4 mg/kg on Day 5, and (d) the first treatment dose of 0.8 mg/kg on Day 7.
   8A. The method of any of embodiments 5A-7A, wherein each of the second step-up dose and the third step-up dose is administered between 2 to 4 days after the previous dose, or is optionally administered up to 7 days after the previous dose to allow for resolution of adverse reactions; and the first treatment dose is administered between 2-7 days after the third step-up dose.
   9A. The method of any of embodiments 5A-7A, wherein each of the second step-up dose, the third step-up dose and the first treatment dose is administered between 2 to 4 days after the previous dose, or is optionally administered up to 7 days after the previous dose to allow for resolution of adverse reactions.
   10A. The method of any of embodiments 1A-9A further comprising administering pretreatment medications to the subject prior to restarting the planned dosing regimen, wherein the pretreatment medications comprise 16 mg or equivalent of oral or intravenous dexamethasone, 50 mg or equivalent of oral or intravenous diphenhydramine, and 650 mg to 1000 mg, or equivalent, oral or intravenous acetaminophen.
   11A. The method of any of embodiments 1A-10A, wherein the GPRC5DxCD3 bispecific antibody comprises a GPRC5D binding domain comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6, and a CD3 binding domain comprising the HCDR1 of SEQ ID NO: 11, the HCDR2 of SEQ ID NO: 12, the HCDR3 of SEQ ID NO: 13, the LCDR1 of SEQ ID NO: 14, the LCDR2 of SEQ ID NO: 15 and the LCDR3 of SEQ ID NO: 16.
   12A. The method of any of embodiments 1A-11A, wherein the GPRC5DxCD3 bispecific antibody comprises a GPRC5D binding domain comprising a heavy chain variable region (VH) having the amino acid sequence of SEQ ID NO: 7 and a light chain variable region (VL) having the amino acid sequence of SEQ ID NO: 8, and the CD3 binding domain comprises a heavy chain variable region (VH) having the amino acid sequence of SEQ ID NO: 17 and a light chain variable region (VL) having the amino acid sequence of SEQ ID NO: 18.
   13A. The method of any of embodiments 1A-12A, wherein the GPRC5DxCD3 bispecific antibody is an IgG1, an IgG2, an IgG3 or an IgG4 isotype.
   14A. The method of any of embodiments 1A-13A, wherein the GPRC5DxCD3 bispecific antibody is an IgG4 isotype.
   15A. The method of any of embodiments 1A-14A, wherein the GPRC5DxCD3 bispecific antibody comprises one or more substitutions in its Fc region.
   16A. The method of any of embodiments 1A-15A, wherein the GPRC5DxCD3 bispecific antibody is an IgG4 isotype and comprises Proline/Alanine/Alanine substitutions at amino acid positions 228/234/235, respectively, in its Fc region (according to EU index numbering).
   17A. The method of any of embodiments 1A-16A, wherein the GPRC5DxCD3 bispecific antibody is an IgG4 isotype and comprises F405L and R409K substitutions in its Fc region (according to EU index numbering).
   18A. The method of any of embodiments 1A-17A, wherein the Fc region of the GPRC5D-binding arm comprises Proline/Alanine/Alanine substitutions at amino acid positions 228/234/235, respectively (according to EU index numbering).
   19A. The method of any of embodiments 1A-18A, wherein the Fc region of the CD3-binding arm comprises Proline/Alanine/Alanine substitutions at amino acid positions 228/234/235, respectively, in addition to F405L and R409K substitutions (according to EU index numbering).
   20A. The method of any of embodiments 1A-19A, wherein the GPRC5DxCD3 bispecific antibody comprises a first heavy chain (HC1) having the amino acid sequence of SEQ ID NO: 9, a first light chain (LC1) having the amino acid sequence of SEQ ID NO: 10, a second heavy chain (HC2) having the amino acid sequence of SEQ ID NO: 19 and a second light chain (LC2) having the amino acid sequence of SEQ ID NO: 20.
   21A. The method of any of embodiments 1A-19A, wherein the GPRC5DxCD3 bispecific antibody comprises a first heavy chain (HC1) having at least 90% identity to the amino acid sequence of SEQ ID NO: 9, a first light chain (LC1) having at least 90% identity to the amino acid sequence of SEQ ID NO: 10, a second heavy chain (HC2) having at least 90% identity to the amino acid sequence of SEQ ID NO: 19 and a second light chain (LC2) having at least 90% identity to the amino acid sequence of SEQ ID NO: 20.
   22A. The method of any of embodiments 1A-19A, wherein the GPRC5DxCD3 bispecific antibody comprises a first heavy chain (HC1) having at least 95% identity to the amino acid sequence of SEQ ID NO: 9, a first light chain (LC1) having at least 95% identity to the amino acid sequence of SEQ ID NO: 10, a second heavy chain (HC2) having at least 95% identity to the amino acid sequence of SEQ ID NO: 19 and a second light chain (LC2) having at least 95% identity to the amino acid sequence of SEQ ID NO: 20.
   23A. The method of any of embodiments 1A-19A, wherein the GPRC5DxCD3 bispecific antibody comprises a first heavy chain (HC1) having at least 98% identity to the amino acid sequence of SEQ ID NO: 9, a first light chain (LC1) having at least 98% identity to the amino acid sequence of SEQ ID NO: 10, a second heavy chain (HC2) having at least 98% identity to the amino acid sequence of SEQ ID NO: 19 and a second light chain (LC2) having at least 98% identity to the amino acid sequence of SEQ ID NO: 20.
   24A. The method of any of embodiments 1A-19A, wherein the GPRC5DxCD3 bispecific antibody comprises a first heavy chain (HC1) having at least 99% identity to the amino acid sequence of SEQ ID NO: 9, a first light chain (LC1) having at least 99% identity to the amino acid sequence of SEQ ID NO: 10, a second heavy chain (HC2) having at least 99% identity to the amino acid sequence of SEQ ID NO: 19 and a second light chain (LC2) having at least 99% identity to the amino acid sequence of SEQ ID NO: 20.
   25A. The method of any of embodiments 1A-20A, wherein the GPRC5DxCD3 bispecific antibody is talquetamab.
   26A. The method of any of embodiments 1A-25A, wherein the subject has relapsed or refractory multiple myeloma.
   27A. The method of any of embodiments 1A-26A, wherein the subject has relapsed and refractory multiple myeloma and has received at least 3 prior therapies, including an immunomodulatory agent, a proteasome inhibitor, and an anti-CD38 antibody and has demonstrated disease progression on the last therapy.
   28A. The method of any of embodiments 1A-26A, wherein the subject has relapsed or refractory multiple myeloma and has received at least four prior lines of therapy, including a proteasome inhibitor, an immunomodulatory agent and an anti-CD38 monoclonal antibody.
   29A. The method of any of embodiments 1A-28A, wherein each dose of 0.01 mg/kg and each dose of 0.06 mg/kg is administered as 2 mg/mL of the bispecific antibody in solution for injection.
   30A. The method of any of embodiments 1A-29A, wherein each dose of 0.4 mg/kg is administered as 40 mg/mL of the bispecific antibody.
   31A. The method of any of embodiments 1A-30A, wherein each dose of 0.8 mg/kg is administered as 40 mg/mL of the bispecific antibody.
   32A. The method of any of embodiments 1A-31A, wherein the GPRC5DxCD3 bispecific antibody is an approved product.
   33A. The method of embodiment 32A, wherein the approved product is a biosimilar.

### EXAMPLES

These examples are provided for illustrative purposes only and not to limit the scope of the claims provided herein.

### Example 1: Approved Drug Product Label

### Highlights of Prescribing Information

These highlights do not include all the information needed to use TALVEY safely and effectively. See full prescribing information for TALVEY.

TALVEY™ talquetamab-tgvs) injection, for subcutaneous use

Initial U.S. Approval 2023

### Warning: Cytokine Release Syndrome and Neurologic Toxicity, Including Immune Effector Cell-Associated Neurotoxicity Syndrome

See full prescribing information for complete boxed warning. Cytokine release syndrome (CRS), including life-threatening or fatal reactions, can occur in patients receiving TALVEY. Initiate TALVEY treatment with step-up dosing to reduce the risk of CRS. Withhold TALVEY until CRS resolves or permanently discontinue based on severity. (2.2, 2.5, 5.1)
Neurologic toxicity, including immune effector cell-associated neurotoxicity syndrome (ICANS), and serious and life-threatening or fatal reactions, can occur in patients receiving TALVEY. Monitor patients for signs or symptoms of neurologic toxicity, including ICANS, during treatment. Withhold or permanently discontinue TALVEY based on severity. (2.5, 5.2)
TALVEY is available only through a restricted program called the TECVAYLI and TALVEY Risk Evaluation and Mitigation Strategy (REMS). (5.3)

### Indications and Usage

TALVEY is a bispecific GPRC5D-directed CD3 T-cell engager indicated for the treatment of adult patients with relapsed or refractory multiple myeloma who have received at least four prior lines of therapy, including a proteasome inhibitor, an immunomodulatory agent and an anti-CD38 monoclonal antibody.

This indication is approved under accelerated approval based on response rate and durability of response. Continued approval for this indication may be contingent upon verification and description of clinical benefit in a confirmatory trial(s). (1)

### Dosage and Administration

For subcutaneous injection. (2.2)

Patients should be hospitalized for 48 hours after all doses within the step-up dosing schedule. (2.1)

Administer pretreatment medications as recommended. (2.3)

See Full Prescribing Information for instructions on preparation and administration. (2.6)

TALVEY Weekly Dosing Schedule (2.2)

| Dosing schedule | Day | Dose^{a} | |
|---|---|---|---|
| Step-up dosing | Day 1 | Step-up dose 1 | 0.01 mg/kg |
| schedule | Day 4^{b} | Step-up dose 2 | 0.06 mg/kg |
| | Day 7^{b} | First treatment | 0.4 mg/kg |
| | | dose | |
| Weekly dosing | One week after first | Subsequent | 0.4 mg/kg once |
| schedule | treatment dose and | treatment doses | weekly |
| | weekly thereafter^{c} | | |

^{a}Based on actual body weight.
^{b}Dose may be administered between 2 to 4 days after the previous dose and may be given up to 7 days after the previous dose to allow for resolution of adverse reactions.
^{c}Maintain a minimum of 6 days between weekly doses.

TALVEY Biweekly (Every 2 Weeks) Dosing Schedule (2.2)

| Dosing schedule | Day | Dose^{a} | |
|---|---|---|---|
| Step-up dosing | Day 1 | Step-up dose 1 | 0.01 mg/kg |
| schedule | Day 4 [symbol] | Step-up dose 2 | 0.06 mg/kg |
| | Day 7 [symbol] | Step-up dose 3 | 0.4 mg/kg |
| | Day 10^{c} | First treatment | 0.8 mg/kg |
| | | dose | |
| Biweekly (every | Two weeks after | Subsequent | 0.8 mg/kg every |
| 2 weeks) dosing | first treatment dose | treatment doses | 2 weeks |
| schedule | and every 2 weeks | | |
| | thereafter^{d} | | |

^{a}Based on actual body weight.
^{ba}Dose may be administered between 2 to 4 days after the previous dose and may be given up to 7 days after the previous dose to allow for resolution of adverse reactions.
^{c}Dose may be administered between 2 to 7 days after step-up dose 3.
^{d}Maintain a minimum of 12 days between biweekly (every 2 weeks) doses. [symbol]
indicates data missing or illegible when filed

### Dosage Forms and Strengths

### Injection

3 mg/1.5 mL (2 mg/mL) in a single-dose vial (3)
40 mg/mL in a single-dose vial (3)

### Contraindications

None. (4)

### Warnings and Precautions

Oral Toxicity and Weight Loss: Monitor for oral toxicity and weight loss. Withhold or permanently discontinue based on severity. (5.4)

Infections: Can cause serious, life-threatening, or fatal infections. Monitor for signs and symptoms of infection; treat appropriately. Withhold or consider permanent discontinuation based on severity. (5.5)

Cytopenias: Monitor complete blood counts. (5.6)

Skin Toxicity: Monitor for skin toxicity, including rash progression, for early intervention and treat appropriately. Withhold as recommended based on severity. (5.7)

Hepatotoxicity: Monitor liver enzymes and bilirubin at baseline and during treatment as clinically indicated. Withhold or consider permanent discontinuation based on severity. (5.8)

Embryo-Fetal Toxicity: May cause fetal harm. Advise females of reproductive potential of the potential risk to the fetus and to use effective contraception. (5.9, 8.1, 8.3)

### Adverse Reactions

The most common adverse reactions (≥20%) are pyrexia, CRS, dysgeusia, nail disorder, musculoskeletal pain, skin disorder, rash, fatigue, weight decreased, dry mouth, xerosis, dysphagia, upper respiratory tract infection, diarrhea, hypotension, and headache. (6.1)

The most common Grade 3 or 4 laboratory abnormalities (≥30%) are lymphocyte count decreased, neutrophil count decreased, white blood cell decreased, and hemoglobin decreased. (6.1)

To report SUSPECTED ADVERSE REACTIONS, contact Janssen Biotech, Inc. at 1-800-JANSSEN (1-800-526-7736) or FDA at 1-800-FDA-1088 or www.fda.gov/medwatch.

### Use in Specific Populations

Lactation: Advise not to breastfeed. (8.2)

See 17 for PATIENT COUNSELING INFORMATION and Medication Guide.

Revised: August 2023

### Full Prescribing Information: Contents*

### Warning: Cytokine Release Syndrome and Neurologic Toxicity, Including Immune Effector Cell-Associated Neurotoxicity Syndrome

1 INDICATIONS AND USAGE
2 DOSAGE AND ADMINISTRATION
   2.1 Important Dosing Information
   2.2 Recommended Dosage
   2.3 Recommended Pretreatment Medications
   2.4 Dosage Delays
   2.5 Dosage Modifications for Adverse Reactions
   2.6 Preparation and Administration
3 DOSAGE FORMS AND STRENGTHS
4 CONTRAINDICATIONS
5 WARNINGS AND PRECAUTIONS
   5.1 Cytokine Release Syndrome (CRS)
   5.2 Neurologic Toxicity including ICANS
   5.3 TECVAYLI and TALVEY REMS
   5.4 Oral Toxicity and Weight Loss
   5.5 Infections
   5.6 Cytopenias
   5.7 Skin Toxicity
   5.8 Hepatotoxicity
   5.9 Embryo-Fetal Toxicity
6 ADVERSE REACTIONS
   6.1 Clinical Trials Experience
7 DRUG INTERACTIONS
8 USE IN SPECIFIC POPULATIONS
   8.1 Pregnancy
   8.2 Lactation
   8.3 Females and Males of Reproductive Potential
   8.4 Pediatric Use
   8.5 Geriatric Use
11 DESCRIPTION
12 CLINICAL PHARMACOLOGY
   12.1 Mechanism of Action
   12.2 Pharmacodynamics
   12.3 Pharmacokinetics
   12.6 Immunogenicity
13 NONCLINICAL TOXICOLOGY
   13.1 Carcinogenesis, Mutagenesis, Impairment of Fertility
14 CLINICAL STUDIES
16 HOW SUPPLIED/STORAGE AND HANDLING
17 PATIENT COUNSELING INFORMATION
   *Sections or subsections omitted from the full prescribing information are not listed.
FULL PRESCRIBING INFORMATION

### Warning: Cytokine Release Syndrome and Neurologic Toxicity, Including Immune Effector Cell-Associated Neurotoxicity Syndrome

Cytokine release syndrome (CRS), including life-threatening or fatal reactions, can occur in patients receiving TALVEY. Initiate TALVEY treatment with step-up dosing to reduce the risk of CRS. Withhold TALVEY until CRS resolves or permanently discontinue based on severity [see Dosage and Administration (2.2, 2.5), Warnings and Precautions (5.1)].
Neurologic toxicity, including immune effector cell-associated neurotoxicity syndrome (ICANS), and serious and life threatening or fatal reactions, can occur with TALVEY. Monitor patients for signs and symptoms of neurologic toxicity including ICANS during treatment and treat promptly. Withhold or permanently discontinue TALVEY based on severity [see Dosage and Administration (2.5), Warnings and Precautions (5.2)].
Because of the risk of CRS and neurologic toxicity, including ICANS, TALVEY is

### 1 Indications and Usage

TALVEY is indicated for the treatment of adult patients with relapsed or refractory multiple myeloma who have received at least four prior lines of therapy, including a proteasome inhibitor, an immunomodulatory agent and an anti-CD38 monoclonal antibody.

This indication is approved under accelerated approval based on response rate and durability of response [see Clinical Studies (14)]. Continued approval for this indication may be contingent upon verification and description of clinical benefit in a confirmatory trial(s).

### 2 Dosage and Administration

### 2.1 Important Dosing Information

Administer TALVEY subcutaneously according to the step-up dosing schedule in Tables 1 and 2 to reduce the incidence and severity of cytokine release syndrome (CRS) [see Dosage and Administration (2.2)].

Administer pretreatment medications prior to each dose of TALVEY in the step-up dosing schedule as recommended [see Dosage and Administration (2.2, 2.3)].

TALVEY should only be administered by a qualified healthcare professional with appropriate medical support to manage severe reactions such as CRS and neurologic toxicity including immune effector cell-associated neurotoxicity syndrome (ICANS)[see Warnings and Precautions (5.1, 5.2)].

Due to the risk of CRS and neurologic toxicity, including ICANS, patients should be hospitalized for 48 hours after administration of all doses within the TALVEY step-up dosing schedule [see Dosage and Administration (2.5) and Warnings and Precautions (5.1, 5.2)].

### 2.2 Recommended Dosage

For subcutaneous injection.

Administer pretreatment medications prior to each dose of TALVEY in the step-up dosing schedule [see Dosage and Administration (2.3)].

Administer TALVEY subcutaneously on a weekly or biweekly (every 2 weeks) dosing schedule according to Table 1 or Table 2. Continue treatment until disease progression or unacceptable toxicity.

TABLE 1
TALVEY Weekly Dosing Schedule

| Dosing schedule | Day | Doseª | |
|---|---|---|---|
| Step-up dosing | Day 1 | Step-up dose 1 | 0.01 mg/kg |
| schedule | Day 4^{b} | Step-up dose 2 | 0.06 mg/kg |
| | Day 7^{b} | First treatment dose | 0.4 mg/kg |
| Weekly dosing | One week after first | Subsequent | 0.4 mg/kg |
| schedule | treatment dose and | treatment doses | once weekly |
| | weekly thereafter^{c} | | |

^{a}Based on actual body weight.
^{b}Dose may be administered between 2 to 4 days after the previous dose and may be given up to 7 days after the previous dose to allow for resolution of adverse reactions.
^{c}Maintain a minimum of 6 days between weekly doses.

TABLE 2
TALVEY Biweekly (Every 2 Weeks) Dosing Schedule

| Dosing schedule | Day | Doseª | |
|---|---|---|---|
| Step-up dosing | Day 1 | Step-up dose 1 | 0.01 mg/kg |
| schedule | Day 4^{b} | Step-up dose 2 | 0.06 mg/kg |
| | Day 7^{b} | Step-up dose 3 | 0.4 mg/kg |
| | Day 10^{c} | First treatment dose | 0.8 mg/kg |
| Biweekly (every | Two weeks after | Subsequent | 0.8 mg/kg |
| 2 weeks) dosing | first treatment | treatment doses | every 2 weeks |
| schedule | dose and every 2 | | |
| | weeks thereafter^{d} | | |

^{a}Based on actual body weight.
^{b}Dose may be administered between 2 to 4 days after the previous dose and may be given up to 7 days after the previous dose to allow for resolution of adverse reactions.
^{c}Dose may be administered between 2 to 7 days after step-up dose 3.
^{d}Maintain a minimum of 12 days between biweekly (every 2 weeks) doses.

### 2.3 Recommended Pretreatment Medications

Administer the following pretreatment medications 1 to 3 hours before each dose of TALVEY in the step-up dosing schedule to reduce the risk of CRS [see Warnings and Precautions (5.1)].
Corticosteroid (oral or intravenous dexamethasone, 16 mg or equivalent)
Antihistamines (oral or intravenous diphenhydramine, 50 mg or equivalent)
Antipyretics (oral or intravenous acetaminophen, 650 mg to 1,000 mg or equivalent)

Administration of pretreatment medications may be required for subsequent doses for patients who repeat doses within the TALVEY step-up dosing schedule due to dose delays (see Table 3 or Table 4) or for patients who experienced CRS (see Table 5).

### 2.4 Dosage Delays

If a dose of TALVEY is delayed, restart therapy based on the recommendations in Table 3 and Table 4 and resume weekly or biweekly (every 2 weeks) dosing schedule accordingly [see Dosage and Administration (2.1)]; if a dose is delayed by more than 28 days for an adverse reaction, evaluate the benefit-risk of restarting TALVEY. Administer pretreatment medications prior to restarting TALVEY and monitor patients following administration of TALVEY [see Dosage and Administration (2.2)].

TABLE 3
Recommendations for Restarting TALVEY after Dose Delay-Weekly
Dosing Schedule

| | Time from | |
|---|---|---|
| Last Dose | Last Dose | |
| Administered | Administered | TALVEY Recommendation* |
| 0.01 mg/kg | More than 7 | Restart TAL VEY step-up dosing schedule |
| | days | at step-up dose 1 (0.01 mg/kg). |
| 0.06 mg/kg | 8 to 28 days | Repeat step-up dose 2 (0.06 mg/kg) and |
| | | continue TALVEY step-up dosing schedule. |
| | More than | Restart TALVEY step-up dosing schedule |
| | 28 days | at step-up dose 1 (0.01 mg/kg). |
| 0.4 mg/kg | 8 to 28 days | Continue TAL VEY dosing schedule at |
| | | treatment dose (0.4 mg/kg weekly). |
| | 29 to 56 days | Restart TALVEY step-up dosing schedule |
| | | at step-up dose 2 (0.06 mg/kg). |
| | More than | Consider permanent discontinuation. If |
| | 56 days | restarting TALVEY, begin with the step- |
| | | up dosing schedule at step-up dose 1 |
| | | (0.01 mg/kg). |

*Administer pretreatment medications prior to restarting TALVEY. After restarting TALVEY, resume weekly dosing schedule accordingly [see Dosage and Administration (2.2)].

TABLE 4
Recommendations for Restarting TALVEY after Dose Delay-
Biweekly (Every 2 Weeks) Dosing Schedule

| | Time from | |
|---|---|---|
| Last Dose | Last Dose | |
| Administered | Administered | TALVEY Recommendation* |
| 0.01 mg/kg | More than 7 | Restart TALVEY step-up dosing schedule at |
| | days | step-up dose 1 (0.01 mg/kg). |
| 0.06 mg/kg | 8 to 28 days | Repeat step-up dose 2 (0.06 mg/kg) and |
| | | continue TALVEY step-up dosing schedule. |
| | More than 28 | Restart TALVEY step-up dosing schedule at |
| | days | step-up dose 1 (0.01 mg/kg). |
| 0.4 mg/kg | 8 to 28 days | Repeat step-up dose 3 (0.4 mg/kg) and |
| | | continue TALVEY step-up dosing schedule. |
| | 29 to 56 days | Restart TALVEY step-up dosing schedule at |
| | | step-up dose 2 (0.06 mg/kg). |
| | More than 56 | Consider permanent discontinuation. If |
| | days | restarting TALVEY, begin with the step-up |
| | | dosing schedule at step-up dose 1 (0.01 |
| | | mg/kg). |
| 0.8 mg/kg | 15 to 28 days | Continue TALVEY dosing schedule at |
| | | treatment dose (0.8 mg/kg every 2 weeks). |
| | 29 to 56 days | Restart TALVEY step-up dosing schedule at |
| | | step-up dose 3 (0.4 mg/kg). |
| | More than 56 | Consider permanent discontinuation. If |
| | days | restarting TALVEY, begin with the step-up |
| | | dosing schedule at step-up dose 1 (0.01 |
| | | mg/kg). |

*Administer pretreatment medications prior to restarting TALVEY. After restarting TALVEY, resume biweekly (every 2 weeks) dosing schedule accordingly [see Dosage and Administration (2.2)].

### 2.5 Dosage Modifications for Adverse Reactions

Dose delays may be required to manage toxicities related to TALVEY [see Warnings and Precautions (5)].

See Table 5, Table 6, and Table 7 for recommended actions for the management of CRS, ICANS, and neurologic toxicity. See Table 8 for recommended dose modifications for other adverse reactions.

### Cytokine Release Syndrome (CRS)

Identify CRS based on clinical presentation [see Warnings and Precautions (5.1)]. Evaluate and treat other causes of fever, hypoxia, and hypotension. If CRS is suspected, withhold TALVEY until CRS resolves or permanently discontinue based on severity, manage according to the recommendations in Table 5, consider further management per current practice guidelines. Administer supportive therapy for CRS, which may include intensive care for severe or life-threatening CRS. Consider laboratory testing to monitor for disseminated intravascular coagulation (DIC), hematology parameters, as well as pulmonary, cardiac, renal, and hepatic function.

TABLE 5
Recommendations for Management of CRS

| CRS | | |
|---|---|---|
| Grade^{a} | Presenting Symptoms | Actions |
| Grade 1 | Temperature ≥100.4° F. | Withhold TALVEY until CRS |
| | (38° C.)^{b} | resolves.^{c} |
| | | Administer pretreatment |
| | | medication prior to next dose. |
| Grade 2 | Temperature ≥100.4° F. | Withhold TALVEY until CRS |
| | (38° C.)^{b} with either: | resolves. |
| | Hypotension responsive | Administer pretreatment |
| | to fluids and not | medications prior to next dose. |
| | requiring vasopressors, or | Patients should be hospitalized |
| | Oxygen requirement of | for 48 hours following the next |
| | low-flow nasal cannula^{d} | dose.^{c} |
| | or blow-by. | |
| Grade 3 | Temperature ≥100.4° F. | Duration less than 48 hours |
| | (38° C.)^{b} with either: | Withhold TALVEY until CRS |
| | Hypotension requiring | resolves. |
| | one vasopressor, with or | Provide supportive therapy, |
| | without vasopressin, or | which may include intensive care. |
| | Oxygen requirement of | Administer pretreatment |
| | high-flow nasal cannula^{d}, | medications prior to the next dose. |
| | facemask, non-rebreather | Patients should be hospitalized for |
| | mask, or Venturi mask | 48 hours following the next dose.^{c} |
| | | Recurrent or duration greater than |
| | | or equal to 48 hours |
| | | Permanently discontinue |
| | | TALVEY. |
| | | Provide supportive therapy, |
| | | which may include intensive care. |
| Grade 4 | Temperature ≥100.4° F. | Permanently discontinue |
| | (38° C.)^{b} with either: | TALVEY. |
| | Hypotension requiring | Provide supportive therapy, |
| | multiple vasopressors | which may include intensive care. |
| | (excluding vasopressin). | |
| | Or, oxygen requirement | |
| | of positive pressure (e.g., | |
| | continuous positive | |
| | airway pressure [CPAP], | |
| | bilevel positive airway | |
| | pressure [BiPAP], | |
| | intubation, and | |
| | mechanical ventilation) | |

^{a}Based on American Society for Transplantation and Cellular Therapy (ASTCT) grading for CRS (Lee et al 2019).
^{b}Attributed to CRS. Fever may not always be present concurrently with hypotension or hypoxia as it may be masked by interventions such as antipyretics or anticytokine therapy (e.g., corticosteroids).
^{c}See Table 3 and Table 4 for recommendations on restarting TALVEY after dose delays for adverse reactions [see Dosage and Administration (2.4)].
^{d}Low-flow nasal cannula is ≤6 L/min, and high-flow nasal cannula is >6 L/min.

Neurologic Toxicity, including ICANS

At the first sign of neurologic toxicity, including ICANS, withhold TALVEY and consider neurology evaluation. Rule out other causes of neurologic symptoms. Provide supportive therapy, which may include intensive care, for severe or life-threatening neurologic toxicities, including ICANS [see Warnings and Precautions (5.2)]. Manage ICANS and neurologic toxicity according to the recommendations in Table 6 and Table 7 and consider further management per current practice guidelines.

TABLE 6
Recommendations for Management of ICANS

| Grade^{a} | Presenting Symptoms^{b} | Actions |
|---|---|---|
| Grade | ICE score 7-9^{c}, | Withhold TALVEY until ICANS |
| 1 | or depressed level | resolves.^{e} |
| | of consciousness^{d}: | Monitor neurologic symptoms, and |
| | awakens | consider consultation with |
| | spontaneously. | neurologist and other specialists for |
| | | further evaluation and management. |
| | | Consider non-sedating, anti-seizure |
| | | medicines (e.g., levetiracetam) for |
| | | seizure prophylaxis. |
| Grade | ICE score 3-6^{c}, | Withhold TALVEY until ICANS |
| 2 | or depressed level | resolves. |
| | of consciousness^{d}: | Administer dexamethasone^{f} 10 mg |
| | awakens to voice. | intravenously every 6 hours. Continue |
| | | dexamethasone use until resolution to |
| | | Grade 1 or less, then taper. |
| | | Monitor neurologic symptoms and |
| | | consider consultation with neurologist |
| | | and other specialists for further |
| | | evaluation and management. |
| | | Consider non-sedating, anti-seizure |
| | | medicines (e.g., levetiracetam) for |
| | | seizure prophylaxis. |
| | | Patients should be hospitalized for 48 |
| | | hours following the next dose of |
| | | TALVEY [see Dosage and |
| | | Administration (2.1)].^{e} |
| Grade | ICE score 0-2^{c}, | First Occurrence of Grade 3 ICANS: |
| 3 | (If ICE score is 0, | Withhold TALVEY until ICANS |
| | but the patient is | resolves. |
| | arousable (e.g., | Administer dexamethasone^{f} 10 mg |
| | awake with global | intravenously every 6 hours. Continue |
| | aphasia) and able to | dexamethasone use until resolution to |
| | perform assessment) | Grade 1 or less, then taper. |
| | or depressed level | Monitor neurologic symptoms and |
| | of consciousness^{d}: | consider consultation with neurologist |
| | awakens only to | and other specialists for further |
| | tactile stimulus, | evaluation and management. |
| | or seizures^{d}, either: | Consider non-sedating, anti-seizure |
| | any clinical seizure, | medicines (e.g., levetiracetam) for |
| | focal or generalized, | seizure prophylaxis. |
| | that resolves rapidly, | Provide supportive therapy, which |
| | or | may include intensive care. |
| | non-convulsive | Patients should be hospitalized for 48 |
| | seizures one | hours following the next dose of |
| | lectroencephalogram | TALVEY [see Dosage and |
| | (EEG) that resolve | Administration (2.1)].^{e} |
| | with intervention, | Recurrent Grade 3 ICANS: |
| | or raised intracranial | Permanently discontinue TALVEY. |
| | pressure: focal/local | Administer dexamethasone^{f} 10 mg |
| | edema on | intravenously and repeat dose every 6 |
| | neuroimaging^{d}. | hours. Continue dexamethasone use |
| | | until resolution to Grade 1 or less, |
| | | then taper. |
| | | Monitor neurologic symptoms and |
| | | consider consultation with neurologist |
| | | and other specialists for further |
| | | evaluation and management. |
| | | Consider non-sedating, anti-seizure |
| | | medicines (e.g., levetiracetam) for |
| | | seizure prophylaxis. |
| | | Provide supportive therapy, which |
| | | may include intensive care. |
| Grade | ICE score 0^{c} | Permanently discontinue TALVEY. |
| 4 | (Patient is unarousable | Administer dexamethasone^{f} 10 mg |
| | and unable to perform | intravenously and repeat dose every 6 |
| | ICE assessment) | hours. Continue dexamethasone use |
| | or depressed level | until resolution to Grade 1 or less, |
| | of consciousness^{d}: | then taper. |
| | either: | Alternatively, consider administration |
| | patient is unarousable | of methylprednisolone 1,000 mg per |
| | or requires vigorous | day intravenously and continue |
| | or repetitive tactile | methylprednisolone 1,000 mg per |
| | stimuli to arouse, or | day intravenously for 2 or more days. |
| | stupor or coma, | Monitor neurologic symptoms and |
| | or seizures^{d}, either: | consider consultation with |
| | life-threatening | neurologist and other specialists for |
| | prolonged seizure | further evaluation and management. |
| | (>5 minutes), or | Consider non-sedating, anti-seizure |
| | repetitive clinical or | medicines (e.g., levetiracetam) for |
| | electrical seizures | seizure prophylaxis. |
| | without return to | Provide supportive therapy, which |
| | baseline in between, | may include intensive care. |
| | or motor findings^{d}: | |
| | deep focal motor | |
| | weakness such as | |
| | hemiparesis or | |
| | paraparesis, | |
| | or raised intracranial | |
| | pressure/cerebral | |
| | edema^{d}, with signs/ | |
| | symptoms such as: | |
| | diffuse cerebral edema | |
| | on neuroimaging, or | |
| | decerebrate or | |
| | decorticate posturing, | |
| | or | |
| | cranial nerve VI palsy, | |
| | or | |
| | papilledema, or | |
| | Cushing's triad. | |

^{a}Based on ASTCT 2019 grading for ICANS.
^{b}Management is determined by the most severe event, not attributable to any other cause.
^{c}If patient is arousable and able to perform Immune Effector Cell-Associated Encephalopathy (ICE) Assessment, assess: Orientation (oriented to year, month, city, hospital = 4 points); Naming (name 3 objects, e.g., point to clock, pen, button = 3 points); Following Commands (e.g., “show me 2 fingers” or “close your eyes and stick out your tongue” = 1 point); Writing (ability to write a standard sentence = 1 point; and Attention (count backwards from 100 by ten = 1 point). If patient is unarousable and unable to perform ICE Assessment (Grade 4 ICANS) = 0 points.
^{d}Attributable to no other cause.
^{e}See Table 3 and Table 4 for recommendations on restarting TALVEY after dose delays for adverse reactions [see Dosage and Administration (2.4)].
^{f}All references to dexamethasone administration are dexamethasone or equivalent.

TABLE 7
Recommendations for Management of
Neurologic Toxicity (excluding ICANS)

| Adverse | | |
|---|---|---|
| Reaction | Severityª | Actions |
| Neurologic | Grade 1 | Withhold TALVEY until neurologic |
| Toxicityª | | toxicity symptoms resolve or stabilize.^{b} |
| (excluding | Grade 2 | Withhold TALVEY until neurologic |
| ICANS) | Grade 3 | toxicity symptoms improve to Grade |
| | (First | 1 or less.^{b} |
| | occurrence) | Provide supportive therapy. |
| | Grade 3 | Permanently discontinue TALVEY. |
| | (Recurrent) | Provide supportive therapy, which may |
| | Grade 4 | include intensive care. |

^{a}Based on National Cancer Institute Common Terminology Criteria for Adverse Events (NCI CTCAE), version 4.03.
^{b}See Table 3 and Table 4 for recommendations on restarting TALVEY after dose delays for adverse reactions [see Dosage and Administration (2.4)].

### Other Adverse Reactions

The recommended dose modifications for other adverse reactions are provided in Table 8.

TABLE 8
Recommended Dose Modifications for Other Adverse Reactions

| Adverse Reaction | Severity | Dose Modification |
|---|---|---|
| Oral Toxicity and | Grade | Provide supportive care. |
| Weight Loss [see | 1-2 | Consider withholding TALVEY if not |
| Warnings and | | responsive to supportive care.ª |
| Precautions (5.4)] | Grade 3 | Withhold TALVEY until resolution to |
| | | Grade 1 or better and provide |
| | | supportive care.ª |
| | Grade 4 | Permanently discontinue TALVEY. |
| Infections [see | All | Withhold TALVEY in the step-up phase |
| Warnings and | Grades | in patients until infection resolves. |
| Precautions (5.5)] | Grade 3 | Withhold TALVEY during the treatment |
| | | phase until infection improves to Grade 1 |
| | | or better within 28 days.^{b} |
| | Grade 4 | Consider permanent discontinuation of TALVEY. |
| | | If TALVEY is not permanently discontinued, |
| | | withhold subsequent treatment doses of |
| | | TALVEY (i.e., doses administered after |
| | | TALVEY step-up dosing schedule) |
| | | until adverse reaction improves to |
| | | Grade 1 or better.^{b} |
| Cytopenias [see | Absolute | Withhold TALVEY until absolute neutrophil |
| Warnings and | neutrophil count | count is 0.5 × 10⁹/L or higher.ª |
| Precautions (5.6)] | less than | |
| | 0.5 × 10⁹/L | |
| | Febrile | Withhold TALVEY until absolute neutrophil |
| | neutropenia | count is 1.0 × 10⁹/L or higher and fever |
| | | resolves.ª |
| | Hemoglobin less | Withhold TALVEY until hemoglobin is 8 |
| | than 8 g/dL | g/dL or higher.ª |
| | Platelet count less | Withhold TALVEY until platelet count is |
| | than 25,000/mcL | 25,000/mcL or higher and no evidence of |
| | Platelet count | bleeding.ª |
| | between | |
| | 25,000/mcL and | |
| | 50,000/mcL with | |
| | bleeding | |
| Skin Reactions | Grade 3-4 | Withhold TALVEY until adverse reaction |
| [see Warnings and | | improves to Grade 1 or baseline.ª |
| Precautions (5.7)] | | |
| Other Non- | Grade 3 | Withhold TALVEY until adverse reaction |
| hematologic | | improves to Grade 1 or baseline.ª |
| Adverse Reactions^{c} | Grade 4 | Consider permanent discontinuation of TALVEY. |
| [see Warnings and | | If TALVEY is not permanently discontinued, |
| Precautions (5.8) | | withhold subsequent treatment doses of |
| and Adverse | | TALVEY (i.e., doses administered after |
| Reactions (6.1)] | | TALVEY step-up dosing schedule) until |
| | | adverse reaction improves to Grade 1 or less.ª |

^{a}See Table 3 and Table 4 for recommendations on restarting TALVEY after dose delays for adverse reactions [see Dosage and Administration (2.4)].
^{b}For Grade 3 or 4 infection, if TALVEY is withheld for more than 28 days, restart step-up dosing when infection improves to Grade 1 or better.
^{c}Based on National Cancer Institute Common Terminology Criteria for Adverse Events (NCI-CTCAE), Version 4.03.

### 2.6 Preparation and Administration

Administer TALVEY via subcutaneous injection by a healthcare provider.

TALVEY should be administered by a healthcare provider with adequate medical personnel and appropriate medical equipment to manage severe reactions, including CRS and neurologic toxicity, including ICANS [see Warnings and Precautions (5.1, 5.2)].

TALVEY 3 mg/1.5 mL (2 mg/mL) vial and TALVEY 40 mg/mL vial are supplied as ready-to-use solution for injection that do not need dilution prior to administration.

Do not combine TALVEY vials of different concentrations to achieve treatment dose. Use aseptic technique to prepare and administer TALVEY.

### Preparation

Refer to the following reference tables for the preparation of TALVEY.
Use Table 9 to determine total dose, injection volume, and number of vials required based on patient's actual body weight for the 0.01 mg/kg dose using TALVEY 3 mg/1.5

TABLE 9
0.01 mg/kg Dose: Injection Volumes using
TALVEY 3 mg/1.5 mL (2 mg/mL) Vial

| | | | Volume | Number |
|---|---|---|---|---|
| | Body | Total | of | of Vials |
| | Weight | Dose | Injection | (1 vial = |
| 0.01 mg/kg | (kg) | (mg) | (mL) | 1.5 mL) |
| Dose | 35 to 39 | 0.38 | 0.19 | 1 |
| | 40 to 45 | 0.42 | 0.21 | 1 |
| | 46 to 55 | 0.5 | 0.25 | 1 |
| | 56 to 65 | 0.6 | 0.3 | 1 |
| | 66 to 75 | 0.7 | 0.35 | 1 |
| | 76 to 85 | 0.8 | 0.4 | 1 |
| | 86 to 95 | 0.9 | 0.45 | 1 |
| | 96 to 105 | 1 | 0.5 | 1 |
| | 106 to 115 | 1.1 | 0.55 | 1 |
| | 116 to 125 | 1.2 | 0.6 | 1 |
| | 126 to 135 | 1.3 | 0.65 | 1 |
| | 136 to 145 | 1.4 | 0.7 | 1 |
| | 146 to 155 | 1.5 | 0.75 | 1 |
| | 156 to 160 | 1.6 | 0.8 | 1 |

Use Table 10 to determine total dose, injection volume, and number of vials required based on patient's actual body weight for the 0.06 mg/kg dose using TALVEY 3 mg/1.5 mL (2 mg/mL) vial.

TABLE 10
0.06 mg/kg Dose: Injection Volumes using
TALVEY 3 mg/1.5 mL (2 mg/mL) Vial

| | | | Volume | Number |
|---|---|---|---|---|
| | Body | Total | of | of Vials |
| 0.06 mg/kg | Weight | Dose | Injection | (1 vial = |
| Dose | (kg) | (mg) | (mL) | 1.5 mL) |
| | 35 to 39 | 2.2 | 1.1 | 1 |
| | 40 to 45 | 2.6 | 1.3 | 1 |
| | 46 to 55 | 3 | 1.5 | 1 |
| | 56 to 65 | 3.6 | 1.8 | 2 |
| | 66 to 75 | 4.2 | 2.1 | 2 |
| | 76 to 85 | 4.8 | 2.4 | 2 |
| | 86 to 95 | 5.4 | 2.7 | 2 |
| | 96 to 105 | 6 | 3 | 2 |
| | 106 to 115 | 6.6 | 3.3 | 3 |
| | 116 to 125 | 7.2 | 3.6 | 3 |
| | 126 to 135 | 7.8 | 3.9 | 3 |
| | 136 to 145 | 8.4 | 4.2 | 3 |
| | 146 to 155 | 9 | 4.5 | 3 |
| | 156 to 160 | 9.6 | 4.8 | 4 |

Use Table 11 to determine total dose, injection volume, and number of vials required based on patient's actual body weight for the 0.4 mg/kg dose using TALVEY 40 mg/mL vial.

TABLE 11
0.4 mg/kg Dose: Injection Volumes using
TALVEY 40 mg/mL Vial

| | | | Volume | Number |
|---|---|---|---|---|
| | Body | Total | of | of Vials |
| 0.4 mg/kg | Weight | Dose | Injection | (1 vial = |
| Dose | (kg) | (mg) | (mL) | 1 mL) |
| | 35 to 39 | 14.8 | 0.37 | 1 |
| | 40 to 45 | 16 | 0.4 | 1 |
| | 46 to 55 | 20 | 0.5 | 1 |
| | 56 to 65 | 24 | 0.6 | 1 |
| | 66 to 75 | 28 | 0.7 | 1 |
| | 76 to 85 | 32 | 0.8 | 1 |
| | 86 to 95 | 36 | 0.9 | 1 |
| | 96 to 105 | 40 | 1 | 1 |
| | 106 to 115 | 44 | 1.1 | 2 |
| | 116 to 125 | 48 | 1.2 | 2 |
| | 126 to 135 | 52 | 1.3 | 2 |
| | 136 to 145 | 56 | 1.4 | 2 |
| | 146 to 155 | 60 | 1.5 | 2 |
| | 156 to 160 | 64 | 1.6 | 2 |

Use Table 12 to determine total dose, injection volume, and number of vials required based on patient's actual body weight for the 0.8 mg/kg dose using TALVEY 40 mg/mL vial.

TABLE 12
0.8 mg/kg Dose: Injection Volumes using
TALVEY 40 mg/mL Vial

| | | | Volume | Number |
|---|---|---|---|---|
| | Body | Total | of | of Vials |
| 0.8 mg/kg | Weight | Dose | Injection | (1 vial = |
| Dose | (kg) | (mg) | (mL) | 1 mL) |
| | 35 to 39 | 29.6 | 0.74 | 1 |
| | 40 to 45 | 34 | 0.85 | 1 |
| | 46 to 55 | 40 | 1 | 1 |
| | 56 to 65 | 48 | 1.2 | 2 |
| | 66 to 75 | 56 | 1.4 | 2 |
| | 76 to 85 | 64 | 1.6 | 2 |
| | 86 to 95 | 72 | 1.8 | 2 |
| | 96 to 105 | 80 | 2 | 2 |
| | 106 to 115 | 88 | 2.2 | 3 |
| | 116 to 125 | 96 | 2.4 | 3 |
| | 126 to 135 | 104 | 2.6 | 3 |
| | 136 to 145 | 112 | 2.8 | 3 |
| | 146 to 155 | 120 | 3 | 3 |
| | 156 to 160 | 128 | 3.2 | 4 |

Parenteral drug products should be inspected visually for particulate matter and discoloration prior to administration, whenever solution and container permit. Check that the TALVEY solution for injection is colorless to light yellow. Do not use if the solution is discolored, cloudy, or if foreign particles are present.
Remove the appropriate strength TALVEY vial(s) from refrigerated storage [2° C. to 8° C. (36° F. to 46° F.)] and equilibrate to ambient temperature [15° C. to 30° C. (59° F. to 86° F.)] for at least 15 minutes. Do not warm TALVEY in any other way.
Once equilibrated, gently swirl the vial for approximately 10 seconds to mix. Do not shake.
Withdraw the required injection volume of TALVEY from the vial(s) into an appropriately sized syringe using a transfer needle.
Each injection volume should not exceed 2 mL. Divide doses requiring greater than 2 mL equally into multiple syringes.
TALVEY is compatible with stainless steel injection needles and polypropylene or polycarbonate syringe material.
Replace the transfer needle with an appropriately sized needle for injection.

### Administration

Inject the required volume of TALVEY into the subcutaneous tissue of the abdomen (preferred injection site). Alternatively, TALVEY may be injected into the subcutaneous tissue at other sites (e.g., thigh). If multiple injections are required, TALVEY injections should be at least 2 cm apart.
Do not inject into tattoos or scars or areas where the skin is red, bruised, tender, hard or not intact.
Any unused medicinal product or waste material should be disposed in accordance with local requirements.

### Storage

The prepared syringes should be administered immediately. If immediate administration is not possible, store the TALVEY solution refrigerated at 2° C. to 8° C. (36° F. to 46° F.) for up to 24 hours followed by at room temperature of 15° C. to 30° C. (59° F. to 86° F.) for up to 24 hours. Discard if stored for more than 24 hours refrigerated or more than 24 hours at room temperature. If stored in the refrigerator, allow the solution to come to room temperature before administration.

### 3 Dosage Forms and Strengths

### Injection

3 mg/1.5 mL (2 mg/mL) colorless to light yellow solution in a single-dose vial
40 mg/mL colorless to light yellow solution in a single-dose vial

### 4 Contraindications

None.

### Warnings and Precautions

### 5.1 Cytokine Release Syndrome (CRS)

TALVEY can cause cytokine release syndrome, including life-threatening or fatal reactions [see Adverse Reactions (6.1)].

In the clinical trial, CRS occurred in 76% of patients who received TALVEY at the recommended dosages, with Grade 1 CRS occurring in 57% of patients, Grade 2 in 17%, and Grade 3 in 1.5%. Recurrent CRS occurred in 30% of patients. Most events occurred following step-up dose 1 (29%)

or step-up dose 2 (44%) at the recommended dosages. CRS occurred in 33% of patients with step-up dose 3 in the biweekly dosing schedule (N=153). CRS occurred in 30% of patients with the first 0.4 mg/kg treatment dose and in 12% of patients treated with the first 0.8 mg/kg treatment dose. The CRS rate for both dosing schedules combined was less than 3% for each of the remaining doses in Cycle 1 and less than 3% cumulatively from Cycle 2 onward. The median time to onset of CRS was 27 (range: 0.1 to 167) hours from the last dose, and the median duration was 17 (range: 0 to 622) hours. Clinical signs and symptoms of CRS include but are not limited to pyrexia, hypotension, chills, hypoxia, headache, and tachycardia. Potentially life-threatening complications of CRS may include cardiac dysfunction, acute respiratory distress syndrome, neurologic toxicity, renal and/or hepatic failure, and disseminated intravascular coagulation (DIC).

Initiate TALVEY therapy with step-up dosing and administer pre-treatment medications (corticosteroids, antihistamine, and antipyretics) prior to each dose of TALVEY in the step-up dosing schedule to reduce the risk of CRS. Monitor patients following administration accordingly. In patients who experience CRS, pre-treatment medications should be administered prior to the next TALVEY dose [see Dosage and Administration (2.2, 2.3)].

Counsel patients to seek medical attention should signs or symptoms of CRS occur. At the first sign of CRS, immediately evaluate patient for hospitalization and institute treatment with supportive care based on severity and consider further management per current practice guidelines. Withhold TALVEY until CRS resolves or permanently discontinue based on severity [see Dosage and Administration (2.5)].

TALVEY is available only through a restricted program under a REMS [see Warnings and Precautions (5.3)].

5.2 Neurologic Toxicity including ICANS

TALVEY can cause serious, life-threatening, or fatal neurologic toxicity, including ICANS [see Adverse Reactions (6.1)].

In the clinical trial, neurologic toxicity, including ICANS, occurred in 55% of patients who received TALVEY at the recommended dosages, with Grade 3 or 4 neurologic toxicity occurring in 6% of patients. The most frequent neurologic toxicities were headache (20%), encephalopathy (15%), sensory neuropathy (14%), and motor dysfunction (10%).

ICANS was reported in 9% of 265 patients where ICANS was collected and who received TALVEY at the recommended dosages [see Adverse Reactions (6.1)]. Recurrent ICANS occurred in 3% of patients. Most patients experienced ICANS following step-up dose 1 (3%), step-up dose 2 (3%), step-up dose 3 of the biweekly dosing schedule (1.8%), or the initial treatment dose of the weekly dosing schedule (2.6%) (N=156) or the biweekly dosing schedule (3.7%) (N=109). The median time to onset of ICANS was 2.5 (range: 1 to 16) days after the most recent dose with a median duration of 2 (range: 1 to 22) days. The onset of ICANS can be concurrent with CRS, following resolution of CRS, or in the absence of CRS. Clinical signs and symptoms of ICANS may include but are not limited to confusional state, depressed level of consciousness, disorientation, somnolence, lethargy, and bradyphrenia.

Monitor patients for signs and symptoms of neurologic toxicity during treatment. At the first sign of neurologic toxicity, including ICANS, immediately evaluate the patient and provide supportive care based on severity; withhold or permanently discontinue TALVEY based on severity and consider further management per current practice guidelines [see Dosage and Administration (2.5)].

Due to the potential for neurologic toxicity, patients receiving TALVEY are at risk of depressed level of consciousness. Advise patients to refrain from driving or operating heavy or potentially dangerous machinery during the step-up dosing schedule and for 48 hours after completion of the step-up dosing schedule [see Dosage and Administration (2.2)] and in the event of new onset of any neurological symptoms, until symptoms resolve.

TALVEY is available only through a restricted program under a REMS [see Warnings and Precautions (5.3)].

### 5.3 TECVAYLI and TALVEY REMS

TALVEY is available only through a restricted program under a REMS called the TECVAYLI and TALVEY REMS because of the risks of CRS and neurologic toxicity, including ICANS [see Warnings and Precautions (5.1, 5.2)].

Notable requirements of the TECVAYLI and TALVEY REMS include the following:
Prescribers must be certified with the program by enrolling and completing training.
Prescribers must counsel patients receiving TALVEY about the risk of CRS and neurologic toxicity, including ICANS and provide patients with Patient Wallet Card.
Pharmacies and healthcare settings that dispense TALVEY must be certified with the TECVAYLI and TALVEY REMS program and must verify prescribers are certified through the TECVAYLI and TALVEY REMS program.
Wholesalers and distributers must only distribute TALVEY to certified pharmacies.

Further information about the TECVAYLI and TALVEY REMS program is available at www.TEC-TALREMS.com or by telephone at 1-855-810-8064.

### 5.4 Oral Toxicity and Weight Loss

TALVEY can cause oral toxicities, including dysgeusia, dry mouth, dysphagia, and stomatitis [see Adverse Reactions (6.1)].

In the clinical trial, 80% of patients had oral toxicity, with Grade 3 occurring in 2.1% of patients who received TALVEY at the recommended dosages. The most frequent oral toxicities were dysgeusia (49%), dry mouth (34%), dysphagia (23%), and ageusia (18%). The median time to onset of oral toxicity was 15 (range: 1 to 634) days, and the median time to resolution to baseline was 43 (1 to 530) days. Oral toxicity did not resolve to baseline in 65% of patients.

TALVEY can cause weight loss [see Adverse Reactions (6.1)]. In the clinical trial, 62% of patients experienced weight loss, regardless of having an oral toxicity, including 29% of patients with Grade 2 (10% or greater) weight loss and 2.7% of patients with Grade 3 (20% or greater) weight loss. The median time to onset of Grade 2 or higher weight loss was 67 (range: 6 to 407) days, and the median time to resolution was 50 (range: 1 to 403) days. Weight loss did not resolve in 57% of patients who reported weight loss.

Monitor patients for signs and symptoms of oral toxicity. Counsel patients to seek medical attention should signs or symptoms of oral toxicity occur and provide supportive care as per current clinical practice including consultation with a nutritionist. Monitor weight regularly during therapy. Evaluate clinically significant weight loss further. Withhold TALVEY or permanently discontinue based on severity [see Dosage and Administration (2.5)].

### 5.5 Infections

TALVEY can cause serious infections, including life-threatening or fatal infections [see Adverse Reactions (6.1)].

In the clinical trial, serious infections occurred in 16% of patients, with fatal infections in 1.5% of patients. Grade 3 or 4 infections occurred in 17% of patients. The most common serious infections reported were bacterial infection (8%), which included sepsis, and COVID-19 (2.7%).

Monitor patients for signs and symptoms of infection prior to and during treatment with TALVEY and treat appropriately. Administer prophylactic antimicrobials according to local guidelines. Withhold or consider permanent discontinuation of TALVEY as recommended based on severity [see Dosage and Administration (2.5)].

### 5.6 Cytopenias

TALVEY can cause cytopenias, including neutropenia and thrombocytopenia [see Adverse Reactions (6.1)].

In the clinical trial, Grade 3 or 4 decreased neutrophils occurred in 35% of patients, and Grade 3 or 4 decreased platelets occurred in 22% of patients who received TALVEY. The median time to onset for Grade 3 or 4 neutropenia was 22 (range: 1 to 312) days, and the median time to resolution to Grade 2 or lower was 8 (range: 1 to 79) days. The median time to onset for Grade 3 or 4 thrombocytopenia was 12 (range: 2 to 183) days, and the median time to resolution to Grade 2 or lower was 10 (range: 1 to 64) days. Monitor complete blood counts during treatment and withhold TALVEY as recommended based on severity [see Dosage and Administration (2.5)].

### 5.7 Skin Toxicity

TALVEY can cause serious skin reactions, including rash, maculo-papular rash, erythema, and erythematous rash [see Adverse Reactions (6.1)].

In the clinical trial, skin reactions occurred in 62% of patients, with Grade 3 skin reactions in 0.3%. The median time to onset was 25 (range: 1 to 630) days. The median time to improvement to Grade 1 or less was 33 days.

Monitor for skin toxicity, including rash progression. Consider early intervention and treatment to manage skin toxicity. Withhold TALVEY as recommended based on severity [see Dosage and Administration (2.5)].

### 5.8 Hepatotoxicity

TALVEY can cause hepatoxicity. In the clinical trial, elevated ALT occurred in 33% of patients, with Grade 3 or 4 ALT elevation occurring in 2.7%; elevated AST occurred in 31% of patients, with Grade 3 or 4 AST elevation occurring in 3.3%. Grade 3 or 4 elevations of total bilirubin occurred in 0.3% of patients [see Adverse Reactions (6.1)]. Liver enzyme elevation can occur with or without concurrent CRS.

Monitor liver enzymes and bilirubin at baseline and during treatment as clinically indicated. Withhold TALVEY or consider permanent discontinuation of TALVEY based on severity [see Dosage and Administration (2.5)].

### 5.9 Embryo-Fetal Toxicity

Based on its mechanism of action, TALVEY may cause fetal harm when administered to a pregnant woman. Advise pregnant women of the potential risk to the fetus. Advise females of reproductive potential to use effective contraception during treatment with TALVEY and for 3 months after the last dose [see Use in Specific Populations (8.1, 8.3)].

### 6 Adverse Reactions

The following adverse reactions are also described elsewhere in the labeling:
Cytokine Release Syndrome [see Warnings and Precautions (5.1)]
Neurologic Toxicity, including ICANS [see Warnings andPrecautions (5.2)]
Oral Toxicity and Weight Loss [see Warnings and Precautions (5.4)]
Infections [see Warnings and Precautions (5.5)]
Cytopenias [see Warnings and Precautions (5.6)]
Skin Toxicity [see Warnings and Precautions (5.7)]
Hepatotoxicity [see Warnings and Precautions (5.8)]

### 6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

### Relapsed/Refractory Multiple Myeloma

### MonumenTAL-1

The safety of TALVEY was evaluated in 339 adult patients with relapsed or refractory multiple myeloma. Patients treated with the weekly dosing schedule received step-up doses of 0.01 mg/kg and 0.06 mg/kg of TALVEY followed by TALVEY 0.4 mg/kg subcutaneously weekly thereafter. Patients treated with the biweekly (every 2 weeks) dosing schedule received step-up doses of 0.01 mg/kg, 0.06 mg/kg, and 0.3 mg/kg (0.75 times the recommended step-up dose 3) followed by TALVEY 0.8 mg/kg subcutaneously every 2 weeks thereafter. The duration of exposure for the 0.4 mg/kg weekly regimen was 5.9 (range: 0.0 to 25.3) months (N=186) and for the 0.8 mg/kg biweekly (every 2 weeks) regimen, it was 3.7 (range: 0.0 to 17.9) months (N=153).

Serious adverse reactions occurred in 47% of patients who received TALVEY. Serious adverse reactions in ≥2% of patients included CRS (13%), bacterial infection (8%) including sepsis, pyrexia (4.7%), ICANS (3.8%), COVID-19 (2.7%), neutropenia (2.1%), and upper respiratory

tract infection (2.1%).

Fatal adverse reactions occurred in 3.2% of patients who received TALVEY, including COVID-19 (0.6%), dyspnea (0.6%), general physical health deterioration (0.6%), bacterial infection (0.3%) including sepsis, basilar artery occlusion (0.3%), fungal infection (0.3%), infection (0.3%), and pulmonary embolism (0.3%).

Permanent discontinuation of TALVEY due to an adverse reaction occurred in 9% of patients. Adverse reactions which resulted in permanent discontinuation of TALVEY in >1% of patients included ICANS.

Dosage interruptions of TALVEY due to an adverse reaction occurred in 56% of patients. Adverse reactions which required dosage interruption in >5% of patients included pyrexia (150%), CRS (12%), upper respiratory tract infection (9%), COVID-19 (9%), bacterial infection (7) including sepsis, neutropenia (60), and rash (60).

The most common adverse reactions (≥20%) were pyrexia, CRS, dysgeusia, nail disorder, musculoskeletal pain, skin disorder, rash, fatigue, weight decreased, dry mouth, xerosis, dysphagia, upper respiratory tract infection, diarrhea, hypotension, and headache. The most common Grade 3 or 4 laboratory abnormalities (≥30%) were lymphocyte count decreased, neutrophil count decreased, white blood cell decreased, and hemoglobin decreased.

Table 13 summarizes the adverse reactions in MonumenTAL-1.

TABLE 13
Adverse Reactions (≥10%) in Patients
with Relapsed or Refractory Multiple Myeloma
Who Received TALVEY in MonumenTAL-1

| | TALVEY | |
|---|---|---|
| | N = 339 | |
| System Organ Class | Any | Grade |
| | Grade | 3 or 4 |
| Adverse Reaction | (%) | (%) |
| General disorders and | | |
| administration site conditions | | |
| Pyrexia* | 83 | 4.7^{†} |
| Fatigue* | 37 | 3.5^{†} |
| Chills | 19 | 0 |
| Pain* | 18 | 1.8^{†} |
| Edema* | 14 | 0 |
| Injection site reaction* | 13 | 0 |
| Immune system disorders | | |
| Cytokine release syndrome | 76 | 1.5^{†} |
| Gastrointestinal disorders | | |
| Dysgeusia^{1 ‡} | 70 | 0 |
| Dry mouth^{‡} | 34 | 0 |
| Dysphagia | 23 | 0.9^{†} |
| Diarrhea | 21 | 0.9^{†} |
| Stomatitis² | 18 | 1.2^{†} |
| Nausea | 18 | 0 |
| Constipation | 16 | 0 |
| Oral disorder³ | 12 | 0 |
| Skin and subcutaneous | | |
| tissue disorders | | |
| Nail disorder⁴ | 50 | 0 |
| Skin disorder⁵ | 41 | 0.3^{†} |
| Rash⁶ | 38 | 3.5^{†} |
| Xerosis⁷ | 30 | 0 |
| Pruritus | 19 | 0.3^{†} |
| Musculoskeletal | | |
| and connective tissue disorders | | |
| Musculoskeletal pain* | 43 | 3.2^{†} |
| Investigations | | |
| Weight decreased | 35 | 1.5^{†} |
| Infections and infestations | | |
| Upper respiratory tract infection* | 22 | 2.7^{†} |
| Bacterial infection including sepsis^{8 #} | 19 | 9 |
| COVID-19* ^{#} | 11 | 2.7 |
| Fungal infection^{9 #} | 10 | 0.6 |
| Vascular disorders | | |
| Hypotension* | 21 | 2.9 |
| Nervous system disorders | | |
| Headache* | 21 | 0.6^{†} |
| Encephalopathy¹⁰ | 15 | 1.8^{†} |
| Sensory neuropathy¹¹ | 14 | 0 |
| Motor dysfunction¹² | 10 | 0.6^{†} |
| Metabolism and nutrition disorders | | |
| Decreased appetite | 19 | 1.2^{†} |
| Respiratory, thoracic | | |
| and mediastinal disorders | | |
| Cough* | 17 | 0 |
| Dyspnea* ^{#} | 11 | 1.8 |
| Hypoxia* | 10 | 1.5^{†} |
| Cardiac disorders | | |
| Tachycardia* | 11 | 0.6^{†} |

Adverse reactions were graded based on CTCAE Version 4.03, with the exception of CRS, which was graded per ASTCT 2019 criteria.
*Includes other related terms.
^{#} Includes fatal outcome(s): COVID-19 (N = 2), dyspnea (N = 2), bacterial infection including sepsis (N = 1), fungal infection (N = 1).
^{†}Only grade 3 adverse reactions occurred.
^{‡}Per CTCAE v4.03, maximum toxicity grade for dysgeusia is 2 and maximum toxicity grade for dry mouth is 3.
¹Dysgeusia: ageusia, dysgeusia, hypogeusia and taste disorder.
²Stomatitis: cheilitis, glossitis, glossodynia, mouth ulceration, oral discomfort, oral mucosal erythema, oral pain, stomatitis, swollen tongue, tongue discomfort, tongue erythema, tongue edema and oropharyngeal pain.
³Oral disorder: oral disorder, oral dysesthesia, oral mucosal exfoliation, oral toxicity and oropharyngeal pain.
⁴Nail disorder: koilonychia, nail bed disorder, nail cuticle fissure, nail discoloration, nail disorder, nail dystrophy, nail hypertrophy, nail pitting, nail ridging, nail toxicity, onychoclasis, onycholysis and onychomadesis.
⁵Skin disorder: palmar-plantar erythrodysesthesia syndrome, palmoplantar keratoderma, skin discoloration, skin exfoliation and skin fissures.
⁶Rash: dermatitis, dermatitis acneiform, dermatitis contact, dermatitis exfoliative, dermatitis exfoliative generalized, erythema, exfoliative rash, rash, rash erythematous, rash macular, rash maculo-papular, rash papular, rash pruritic, rash pustular, rash vesicular and stasis dermatitis.
⁷Xerosis: dry eye, dry skin and xerosis.
⁸Bacterial infection including sepsis: bacteremia, bacterial prostatitis, carbuncle, cellulitis, citrobacter infection, clostridium difficile colitis, clostridium difficile infection, cystitis escherichia, cystitis klebsiella, diverticulitis, enterobacter bacteremia, escherichia pyelonephritis, escherichia sepsis, folliculitis, gastroenteritis escherichia coli, helicobacter gastritis, human ehrlichiosis, klebsiella bacteremia, klebsiella sepsis, moraxella infection, otitis media acute, pitted keratolysis, pneumococcal sepsis, pneumonia, pneumonia streptococcal, pseudomonal bacteremia, pyuria, renal abscess, salmonella sepsis, sepsis, septic shock, skin infection, staphylococcal bacteremia, staphylococcal infection, staphylococcal sepsis, streptococcal bacteremia, tooth abscess, tooth infection, urinary tract infection enterococcal, and urinary tract infection pseudomonal.
⁹Fungal infection: body tinea, candida infection, ear infection fungal, esophageal candidiasis, fungal infection, fungal sepsis, fungal skin infection, genital candidiasis, onychomycosis, oral candidiasis, oral fungal infection, oropharyngeal candidiasis, tinea pedis, vulvovaginal candidiasis, and vulvovaginal mycotic infection.
¹⁰Encephalopathy: agitation, altered state of consciousness, amnesia, aphasia, bradyphrenia, confusional state, delirium, depressed level of consciousness, disorientation, encephalopathy, hallucination, lethargy, memory impairment, mood altered, restlessness, sleep disorder and somnolence.
¹¹Sensory neuropathy: dysesthesia, hyperesthesia, hypoesthesia, hypoesthesia oral, immune-mediated neuropathy, neuralgia, neuropathy peripheral, paresthesia, peripheral sensory neuropathy, polyneuropathy, sciatica and vestibular neuronitis.
¹²Motor dysfunction: dysarthria, dysgraphia, dysmetria, dysphonia, gait disturbance, muscle atrophy, muscle spasms, muscular weakness and tremor.

Clinically relevant adverse reactions reported in <10% of patients who received TALVEY included ICANS and viral infection.

Table 14 summarizes select laboratory abnormalities in MonumenTAL-1.

TABLE 14
Select Laboratory Abnormalities (≥30%) That Worsened from
Baseline in Patients with Relapsed or Refractory Multiple
Myeloma Who Received TALVEY in MonumenTAL-1

| | TALVEY¹ | |
|---|---|---|
| | Any | Grade |
| | Grade | 3 or 4 |
| Laboratory Abnormality | (%) | (%) |
| Hematology | | |
| Lymphocyte count decreased | 90 | 80 |
| White blood cell decreased | 73 | 35 |
| Hemoglobin decreased | 67 | 30 |
| Neutrophil count decreased | 64 | 35 |
| Platelet count decreased | 62 | 22 |
| Chemistry | | |
| Albumin decreased | 66 | 2.1 |
| Alkaline phosphatase increased | 49 | 1.5 |
| Phosphate decreased | 44 | 13 |
| Gamma-glutamyl transferase increased | 38 | 7 |
| Alanine aminotransferase increased | 33 | 2.7 |
| Potassium decreased | 31 | 4.4 |
| Sodium decreased | 31 | 6 |
| Aspartate aminotransferase increased | 31 | 3.3 |

¹The denominator used to calculate the rate varied from 326 to 338 based on the number of patients with a baseline value and at least one post-treatment value.
Laboratory toxicity grades are derived based on the NCI CTCAE (National Cancer Institute Common Terminology Criteria for Adverse Events) Version 4.03.

### 7 Drug Interactions

For certain cytochrome P450 (CYP) substrates, minimal changes in the substrate concentration may lead to serious adverse reactions. Monitor for toxicity or drug concentrations of such CYP substrates when co-administered with TALVEY.

Talquetamab-tgvs causes release of cytokines [see Clinical Pharmacology (12.2)] that may suppress activity of CYP enzymes, resulting in increased exposure of CYP substrates. Increased exposure of CYP substrates is more likely to occur from initiation of the TALVEY step-up dosing schedule up to 14 days after the first treatment dose and during and after CRS [see Warnings and Precautions (5.1)].

### 8 Use in Specific Populations

### 8.1 Pregnancy

### Risk Summary

Based on the mechanism of action, TALVEY may cause fetal harm when administered to a pregnant woman [see Clinical Pharmacology (12.1)]. There are no available data on the use of TALVEY in pregnant women to evaluate for a drug associated risk. No animal reproductive or developmental toxicity studies have been conducted with talquetamab-tgvs.

Talquetamab-tgvs causes T-cell activation and cytokine release; immune activation may compromise pregnancy maintenance. Human immunoglobulin G (IgG) is known to cross the placenta; therefore, TALVEY has the potential to be transmitted from the mother to the developing fetus. Advise women of the potential risk to the fetus.

In the U.S. general population, the estimated background risk of major birth defects and miscarriage in clinically recognized pregnancies is 2% to 4% and 15% to 20%, respectively.

### 8.2 Lactation

### Risk Summary

There is no information regarding the presence of talquetamab-tgvs in human milk, the effect on the breastfed child, or the effect on milk production. Maternal IgG is known to be present in human milk. The effects of local gastrointestinal exposure and limited systemic exposure in the breastfed child to TALVEY are unknown. Because of the potential for serious adverse reactions in a breastfed child, advise women not to breastfeed during treatment with TALVEY and for 3 months after the last dose.

### 8.3 Females and Males of Reproductive Potential

TALVEY may cause fetal harm when administered to a pregnant woman [see Use in Specific Populations (8.1)].

### Pregnancy Testing

Verify pregnancy status of females of reproductive potential prior to initiating TALVEY.

### Contraception

### Females

Advise females of reproductive potential to use effective contraception during treatment with TALVEY and for 3 months after the last dose.

### 8.4 Pediatric Use

The safety and efficacy of TALVEY have not been established in pediatric patients.

### 8.5 Geriatric Use

There were 339 patients in the clinical trial for relapsed or refractory multiple myeloma. Of the total number of TALVEY-treated patients in the study, 178 (53%) patients were 65 years of age and older, while 57 (17%) patients were 75 years of age and older. No overall differences in safety or effectiveness were observed in patients 65 to less than 74 years of age compared to younger patients. There was a higher rate of fatal adverse reactions in patients 75 years of age or older compared to younger patients [see Adverse Reactions (6.1)]. Clinical studies did not include sufficient numbers of patients 75 years of age or over to determine whether they respond differently from younger patients.

### 11 Description

Talquetamab-tgvs is a bispecific GPRC5D-directed CD3 T-cell engager. It is a humanized IgG4 proline, alanine, alanine (IgG4-PAA)-based bispecific antibody produced by Chinese hamster ovary (CHO) cells using recombinant DNA technology. Talquetamab-tgvs consists of an anti-GPRC5D heavy chain and light chain and an anti-CD3 heavy chain and light chain with two interchain disulfide bonds connecting the two arms. The molecular weight of talquetamab-tgvs is 147 kDa.

TALVEY™ (talquetamab-tgvs) injection is a sterile, preservative-free colorless to light yellow solution supplied in a single-dose vial for subcutaneous administration.

Each TALVEY 1.5 mL single-dose vial contains 3 mg of talquetamab-tgvs, edetate disodium (0.027 mg), glacial acetic acid (0.36 mg), polysorbate 20 (0.6 mg), sodium acetate (1.39 mg), sucrose (120 mg), and Water for Injection, USP. The pH is 5.2.

Each TALVEY 1 mL single-dose vial contains 40 mg of talquetamab-tgvs, edetate disodium (0.018 mg), glacial acetic acid (0.24 mg), polysorbate 20 (0.4 mg), sodium acetate (0.90 mg), sucrose (80 mg), and Water for Injection, USP. The pH is 5.2.

### 12 Clinical Pharmacology

### 12.1 Mechanism of Action

Talquetamab-tgvs is a bispecific T-cell engaging antibody that binds to the CD3 receptor expressed on the surface of T-cells and G protein-coupled receptor class C group 5 member D (GPRC5D) expressed on the surface of multiple myeloma cells and non-malignant plasma cells, as well as healthy tissues such as epithelial cells in keratinized tissues of the skin and tongue.

In vitro, talquetamab-tgvs activated T-cells caused the release of proinflammatory cytokines and resulted in the lysis of multiple myeloma cells. Talquetamab-tgvs had anti-tumor activity in mouse models of multiple myeloma.

### 12.2 Pharmacodynamics

Serum concentrations of cytokines (IL-6, IL-10, TNF-α, and IFN-γ) and IL-2R were measured before and after administration of each step-up dose, the first three treatment doses at 0.4 mg/kg once weekly, and the first two treatment doses at 0.8 mg/kg every two weeks. Increased concentrations of IL-6, IL-10, and IL-2R were observed during this period.

Higher talquetamab-tgvs exposures (i.e., AUC and Cmax) are associated with higher incidence of some adverse reactions (including oral toxicity, nail toxicity, and skin reactions). The exposure-response relationships for effectiveness and the time course of pharmacodynamic response of talquetamab-tgvs have not been fully characterized.

### 12.3 Pharmacokinetics

The Cmax and AUCtau of talquetamab-tgvs after subcutaneous administration increased proportionally over a dose range of 0.005 to 0.8 mg/kg weekly (0.01 to 2 times the recommended 0.4 mg/kg weekly treatment dose) and 0.8 to 1.2 mg/kg every two weeks (1 to 1.5 times the recommended 0.8 mg/kg every 2 weeks treatment dose). Ninety percent of steady state exposure was achieved 16 weeks after the first treatment dose for both regimens.

The Cmax, Ctrough, Cavg, and accumulation ratios of talquetamab-tgvs are presented in Table 15.

TABLE 15
Pharmacokinetic Parameters of Talquetamab-tgvs Following
the Dose at 16 Weeks After the First Treatment Dose for
the Approved Recommended Subcutaneous Dosages in
Patients with Relapsed or Refractory Multiple Myeloma

| | Talquetamab-tgvs Dosage | |
|---|---|---|
| | 0.4 mg/kg | 0.8 mg/kg |
| Parameter | every week | every 2 weeks |
| Exposure¹ | | |
| Cₘₐₓ (ng/ml) | 2,940 (67%) | 3,410 (63%) |
| C_{trough} (ng/ml) | 2,410 (83%) | 1,930 (103%) |
| C_{avg} (ng/ml) | 2,730 (71%) | 2,770 (72%) |
| Accumulation Ratio² | | |
| Cₘₐₓ | 4.4 | 1.8 |
| C_{trough} | 4.6 | 2.3 |
| C_{avg} | 5.1 | 2.0 |

C_{avg} = Average concentration over the dosing interval; :
Cₘₐₓ = Maximum serum talquetamab-tgvs concentration;
C_{trough} = Serum talquetamab-tgvs concentration prior to next dose
¹Data are presented as geometric mean (coefficient of variation %).
²For the 0.4 mg/kg every week regimen, accumulation ratios are presented as the arithmetic mean of the 17^{th} treatment dose/the first treatment dose.
For the 0.8 mg/kg every 2 weeks regimen, accumulation ratios are presented as the arithmetic mean of the 9^{th} treatment dose/the first treatment dose.

### Absorption

The geometric mean (coefficient of variation [CV]%) bioavailability of talquetamab-tgvs was

59% (22%) when administered subcutaneously.

The median (range) Tmax of talquetamab-tgvs after the first and 17^{th} treatment dose of 0.4 mg/kg weekly were 3.7 (0.9 to 7) days and 2.5 (0.9 to 5.9) days, respectively.

The median (range) Tmax of talquetamab-tgvs after the first and 9^{th} treatment dose of 0.8 mg/kg every 2 weeks were 3.4 (0.8 to 14) days and 3.6 (1 to 7.7) days, respectively.

### Distribution

The geometric mean (CV %) volume of distribution of talquetamab-tgvs was 10.1 L (25%).

### Elimination

Talquetamab-tgvs clearance decreases over time, with a mean (CV %) maximal reduction from the first treatment dose to 16 weeks after the first treatment dose of 40% (56%). The geometric mean (CV %) clearance is 0.90 L/day (63%) at 16 weeks after the first treatment dose. The mean (CV %) terminal half-life was 8.4 (41%) days after the first treatment dose and 12.2 (49%) days at 16 weeks after the first treatment dose.

### Metabolism

Talquetamab-tgvs is expected to be metabolized into small peptides by catabolic pathways.

### Specific Populations

The volume of distribution and clearance of talquetamab-tgvs increased with increasing bodyweight (40 kg to 143 kg).

There were no clinically significant differences in the pharmacokinetics of talquetamab-tgvs based on age (33 to 86 years), sex, race (White, Black or African American), ethnicity (Not Hispanic/Latino, Hispanic/Latino), mild or moderate renal impairment (creatinine clearance [CLcr] by Cockcroft-Gault equation: 30 to 89 mL/min) or mild (total bilirubin less than or equal to upper limit of normal [ULN] with AST greater than ULN or total bilirubin greater than 1 to 1.5 times ULN with any AST) or moderate (total bilirubin greater than 1.5 to less than 3 times ULN with any AST) hepatic impairment. The effects of severe renal impairment (CLcr less than 30 mL/min) or severe hepatic impairment (total bilirubin greater than 3 times ULN with any AST) on the pharmacokinetics of talquetamab-tgvs are unknown.

### 12.6 Immunogenicity

The observed incidence of anti-drug antibodies is highly dependent on the sensitivity and specificity of the assay. Differences in assay methods preclude meaningful comparisons of the incidence of anti-drug antibodies in the studies described below with the incidence of anti-drug antibodies in other studies, including those of talquetamab-tgvs or of other talquetamab products.

During treatment in MonumenTAL-1 (up to 25 months), 45/177 (25%) patients treated with subcutaneous TALVEY 0.4 mg/kg weekly (median follow-up 5.7 months) and 24/130 (18%) patients treated with subcutaneous TALVEY 0.8 mg/kg every 2 weeks (median follow-up 3.1 months) developed anti-talquetamab-tgvs antibodies. There was no identified clinically significant effect of anti-talquetamab-tgvs antibodies on the pharmacokinetics, pharmacodynamics, safety, or effectiveness of TALVEY.

### 13 Nonclinical Toxicology

### 13.1 Carcinogenesis, Mutagenesis, Impairment of Fertility

No carcinogenicity or genotoxicity studies have been conducted with talquetamab-tgvs. No studies have been conducted to evaluate the effects of talquetamab-tgvs on fertility.

### 14 Clinical Studies

The efficacy of TALVEY monotherapy was evaluated in patients with relapsed or refractory multiple myeloma in a single-arm, open-label, multicenter study, MMY1001 (MonumenTAL-1) (NCT03399799, NCT04634552). The study included patients who had previously received at least three prior systemic therapies, including a proteasome inhibitor, an immunomodulatory agent, and an anti-CD38 monoclonal antibody. The study excluded patients who experienced T-cell redirection therapy within 3 months, prior Grade 3 or higher CRS related to any T-cell redirection therapy, an autologous stem cell transplant within the past 12 weeks, an allogenic stem cell transplant within the past 6 months, Eastern Cooperative Oncology Group (ECOG) performance score of 3 or higher, stroke or seizure within the past 6 months, CNS involvement or clinical signs of meningeal involvement of multiple myeloma, and plasma cell leukemia, active or documented history of autoimmune disease (exception of vitiligo, resolved childhood atopic dermatitis, resolved Grave's Disease that is euthyroid based on clinical and laboratory testing).

Patients treated with the weekly dosing schedule received step-up doses of 0.01 mg/kg and 0.06 mg/kg of TALVEY followed by TALVEY 0.4 mg/kg subcutaneously weekly thereafter.

Patients treated with the biweekly (every 2 weeks) dosing schedule received step-up doses of 0.01 mg/kg, 0.06 mg/kg, and 0.3 mg/kg (0.75 times the recommended step-up dose 3) of TALVEY followed by TALVEY 0.8 mg/kg subcutaneously biweekly, thereafter. Patients on both dosing schedules were treated until disease progression or unacceptable toxicity.

The efficacy results from the 187 patients treated with TALVEY who were not exposed to prior T cell redirection therapy and who had received at least 4 prior lines of therapy are presented below; of these patients, the median age was 67 (range: 38 to 86) years, 57% were male, 90% were White, 5% were Black or African American, 3% were Asian, and 8% were Hispanic. Patients had received a median of 5 (range: 4 to 13) prior lines of therapy, and 78% had received prior autologous stem cell transplantation (ASCT). Ninety-four percent (94%) of patients were refractory to their last therapy, and 73% were refractory to a proteasome inhibitor, immunomodulatory agent, and anti-CD38 antibody. The International Staging System (ISS) at study entry was Stage I in 44%, Stage II in 34%, and Stage III in 22% of patients. High-risk cytogenetic factors (presence of t(4:14), t(14:16), and/or del(17p)) were present in 29% of patients; baseline cytogenetic data were not available in 11% of patients. Twenty-two percent (22%) of patients had extramedullary plasmacytomas.

Efficacy was based on overall response rate (ORR) and duration of response (DOR) as assessed by an Independent Review Committee using IMWG criteria. The median duration of follow-up from first response among responders receiving TALVEY 0.4 mg/kg weekly was 13.8 (range: 0.8 to 15.4) months.

TABLE 16
Efficacy Results for MMY1001 (MonumenTAL-1)
in Patients Receiving 0.4 mg/kg Weekly TALVEY

| | 0.4 mg/kg |
|---|---|
| | Weekly |
| | (N = 100) |
| Overall response rate | 73 (73%) |
| (ORR = sCR + CR + VGPR + PR) | |
| 95% CI | (63.2%, 81.4%) |
| Stringent complete response (sCR) | 26% |
| Complete response (CR) | 9% |
| Very good partial response (VGPR) | 22% |
| Partial response (PR) | 16% |
| Duration of Response (DOR) | |
| Median DOR (95% CI) (months) | 9.5 (6.5, NE) |

CI = confidence interval;
MRD = minimal residual disease;
NE = not estimable

The median duration of follow-up from first response among responders receiving TALVEY 0.8 mg/kg biweekly was 5.9 (range: 0 to 9.5) months; an estimated 85% of responders maintained response for at least 9 months.

TABLE 17
Efficacy Results for MMY1001 (MonumenTAL-1) in Patients
Receiving 0.8 mg/kg Biweekly (Every 2 Weeks) TALVEY

| | 0.8 mg/kg Biweekly |
|---|---|
| | (Every 2 Weeks) |
| | (N = 87) |
| Overall response rate | 65 (73.6%) |
| (ORR = sCR + CR + VGPR + PR) | |
| 95% CI | (63.0%, 82.4%) |
| Stringent complete response (sCR) | 20% |
| Complete response (CR) | 13% |
| Very good partial response (VGPR) | 25% |
| Partial response (PR) | 16% |
| Duration of Response (DOR) | |
| Median DOR (95% CI) (months) | NE |

CI = confidence interval;
MRD = minimal residual disease;
NE = not estimable

The median time to first response was 1.2 (range: 0.2 to 10.9) months and 1.3 (range: 0.2 to 9.2) months for 0.4 mg/kg weekly and 0.8 mg/kg biweekly (every 2 weeks), respectively.

Thirty-two patients were exposed to prior T cell redirection therapy and had received at least 4 prior lines of therapy, including a proteasome inhibitor, an immunomodulatory agent, and an anti-CD38 monoclonal antibody received TALVEY at the 0.4 mg/kg weekly dose. Patients had received a median of 6 (range: 4 to 15) prior therapies, with 81% exposed to CAR-T cell therapy and 25% exposed to a bispecific antibody. Ninety-four percent of patients were exposed to prior T cell redirection therapy directed at BCMA. The ORR per IRC assessment was 72% (95% CI: 53%, 86%). With a median duration of follow-up of 10.4 months, an estimated 59% of responders maintained response for at least 9 months.

### 16 how Supplied/Storage and Handling

TALVEY™ (talquetamab-tgvs) injection is a sterile, preservative-free, colorless to light yellow solution supplied as follows:
One 3 mg/1.5 mL (2 mg/mL) single-dose vial in a carton: NDC: 57894-469-01
One 40 mg/mL single-dose vial in a carton: NDC: 57894-470-01

Store refrigerated at 2° C. to 8° C. (36° F. to 46° F.) in original carton to protect from light. Do not freeze.

### 17 Patient Counseling Information

Advise the patient to read the FDA-approved patient labeling (Medication Guide).

### Cytokine Release Syndrome (CRS)

Discuss the signs and symptoms associated with CRS including, but not limited to, pyrexia, hypotension, chills, hypoxia, headache, and tachycardia. Counsel patients to seek medical attention should signs or symptoms of CRS occur. Advise patients that they should be hospitalized for 48 hours after administration of all doses within the TALVEY step-up dosing schedule [see Dosage and Administration (2.1, 2.5), Warnings and Precautions (5.1)].

### Neurologic Toxicity, Including ICANS

Discuss the signs and symptoms associated with neurologic toxicity, including ICANS including headache, encephalopathy, sensory neuropathy, motor dysfunction, ICANS, confusional state, depressed level of consciousness, disorientation, somnolence, lethargy, and bradyphrenia. Counsel patients to seek medical attention should signs or symptoms of ICANS occur. Advise patients to

refrain from driving or operating heavy or potentially dangerous machinery during the step-up dosing schedule and for 48 hours after completion of the step-up dosing schedule and in the event of new onset of any neurologic toxicity symptoms, until symptoms resolve [see Dosage and Administration (2.2, 2.5), Warnings and Precautions (5.2)].

### TECVAYLI and TALVEY REMS

TALVEY is available only through a restricted program called the TECVAYLI and TALVEY REMS. Inform patients that they will be given a Patient Wallet Card that they should carry with them at all times and show to all of their healthcare providers. This card describes signs and symptoms of CRS and neurologic toxicity, including ICANS which, if experienced, should prompt the patient to immediately seek medical attention [see Warnings and Precautions (5.3)].

### Oral Toxicity and Weight Loss

Discuss the signs and symptoms of oral toxicities including dysgeusia, dry mouth, dysphagia, and stomatitis. Counsel patients to seek medical attention should signs or symptoms of oral toxicity occur. Advise patients that they may experience weight loss and to report weight loss. Advise patients that they may be referred to a nutritionist for consultation [see Dosage and Administration (2.5), Warnings and Precautions (5.4)].

### Infections

Discuss the signs and symptoms of serious infections [see Dosage and Administration (2.5), Warnings and Precautions (5.5)].

### Cytopenias

Discuss the signs and symptoms associated with neutropenia and thrombocytopenia [see Dosage and Administration (2.5), Warnings and Precautions (5.6)].

### Skin Toxicity

Discuss the signs and symptoms of skin reactions [see Dosage and Administration (2.5), Warnings and Precautions (5.7)].

### Hepatotoxicity

Advise patients that liver enzyme elevations may occur and that they should report symptoms that may indicate liver toxicity, including fatigue, anorexia, right upper abdominal discomfort, dark urine, or jaundice [see Warnings andPrecautions (5.8)].

### Embryo-Fetal Toxicity

Advise pregnant women and females of reproductive potential of the potential risk to a fetus. Advise females of reproductive potential to inform their healthcare provider if they are pregnant or become pregnant. Advise females of reproductive potential to use effective contraception during treatment with TALVEY and for 3 months after the last dose [see Warnings andPrecautions (5.9), Use in Specific Populations (8.1, 8.3)].

### Lactation

Advise women not to breastfeed during treatment with TALVEY and for 3 months after the last dose [see Use in Specific Populations (8.2)].

Manufactured by:
Janssen Biotech, Inc.
Horsham, PA 19044, USA
U.S. License Number 1864
For patent information: www.janssenpatents.com
© 2023 Janssen Pharmaceutical Companies

This Medication Guide has been approved by the U.S. Food and Drug Administration.
Issued: August 2023

The examples and embodiments described herein are for illustrative purposes only and various modifications or changes suggested to persons skilled in the art are to be included within the spirit and purview of this application and scope of the appended claims.

1-39. (canceled)

40. A method of restarting a subject's dosing regimen for a G protein-coupled receptor, class C group 5 member D (GPCR5D) and cluster of differentiation 3 (CD3) (GPRC5DxCD3) bispecific antibody after the subject has initiated a planned dosing regimen for treatment of multiple myeloma, and a delay has occurred between doses of the GPRC5DxCD3 bispecific antibody during the planned dosing regimen, wherein the planned dosing regimen comprises:
(1) a step-up dosing schedule that includes (a) a first step-up dose of 0.01 mg/kg, (b) a second step-up dose of 0.06 mg/kg between 2 to 7 days after the first step-up dose, and (c) a first treatment dose of 0.4 mg/kg between 2 to 7 days after the second step-up dose; and
(2) a weekly dosing schedule that includes subsequent treatment doses of 0.4 mg/kg one week after the first treatment dose and weekly thereafter, with a minimum of 6 days between said treatment doses,
wherein the method of restarting the subject's dosing regimen comprises:
(i) if the last dose administered to the subject was 0.01 mg/kg, and the time from the last dose administered is more than 7 days, then restarting the step-up dosing schedule at the first step-up dose of 0.01 mg/kg and resuming the planned dosing regimen, or
(ii) if the last dose administered to the subject was 0.06 mg/kg, and the time from the last dose administered is 8 to 28 days, then repeating the second step-up dose of 0.06 mg/kg and continuing the step-up dosing schedule (thereby resuming the planned dosing regimen), or
(iii) if the last dose administered to the subject was 0.06 mg/kg, and the time from the last dose administered is more than 28 days, then restarting the step-up dosing schedule at the first step-up dose of 0.01 mg/kg and resuming the planned dosing regimen, or
(iv) if the last dose administered to the subject was 0.4 mg/kg, and the time from the last dose administered is 8 to 28 days, then continuing the planned dosing regimen at treatment doses of 0.4 mg/kg weekly (thereby resuming the planned dosing regimen), or
(v) if the last dose administered to the subject was 0.4 mg/kg, and the time from the last dose administered is 29 to 56 days, then restarting the step-up dosing schedule at the second step-up dose of 0.06 mg/kg and resuming the planned dosing regimen, or
(vi) if the last dose administered to the subject was 0.4 mg/kg, and the time from the last dose administered is more than 56 days, then either discontinuing the planned dosing regimen permanently, or restarting the step-up dosing schedule at the first step-up dose of 0.01 mg/kg and resuming the planned dosing regimen.

41. The method of claim 40, wherein the step-up dosing schedule includes (a) the first step-up dose of 0.01 mg/kg on Day 1, (b) the second step-up dose of 0.06 mg/kg on Day 4, and (c) the first treatment dose of 0.4 mg/kg on Day 7.

42. The method of claim 40, wherein the step-up dosing schedule includes (a) the first step-up dose of 0.01 mg/kg on Day 1, (b) the second step-up dose of 0.06 mg/kg on Day 3, and (c) the first treatment dose of 0.4 mg/kg on Day 5.

43. The method of claim 40, wherein each of the second step-up dose and the first treatment dose is administered between 2 to 4 days after the previous dose, or is optionally administered up to 7 days after the previous dose to allow for resolution of adverse reactions.

44. A method of restarting a subject's dosing regimen for a GPRC5DxCD3 bispecific antibody after the subject has initiated a planned dosing regimen for treatment of multiple myeloma, and a delay has occurred between doses of the GPRC5DxCD3 bispecific antibody during the planned dosing regimen, wherein the planned dosing regimen comprises:
(1) a step-up dosing schedule that includes (a) a first step-up dose of 0.01 mg/kg, (b) a second step-up dose of 0.06 mg/kg between 2 to 7 days after the first step-up dose, (c) a third step-up dose of 0.4 mg/kg between 2 to 7 days after the second step-up dose, and (d) a first treatment dose of 0.8 mg/kg between 2 to 7 days after the third step-up dose; and
(2) a biweekly (every two weeks) dosing schedule that includes subsequent treatment doses of 0.8 mg/kg two weeks after the first treatment dose and every two weeks thereafter, with a minimum of 12 days between said treatment doses,
wherein the method of restarting the subject's dosing regimen comprises:
(i) if the last dose administered to the subject was 0.01 mg/kg, and the time from the last dose administered is more than 7 days, then restarting the step-up dosing schedule at the first step-up dose of 0.01 mg/kg and resuming the planned dosing regimen, or
(ii) if the last dose administered to the subject was 0.06 mg/kg, and the time from the last dose administered is 8 to 28 days, then repeating the second step-up dose of 0.06 mg/kg, continuing the step-up dosing schedule (thereby resuming the planned dosing regimen), or
(iii) if the last dose administered to the subject was 0.06 mg/kg, and the time from the last dose administered is more than 28 days, then restarting the step-up dosing schedule at the first step-up dose of 0.01 mg/kg and resuming the planned dosing regimen, or
(iv) if the last dose administered to the subject was 0.4 mg/kg, and the time from the last dose administered is 8 to 28 days, then repeating the third step-up dose of 0.4 mg/kg and continuing the step-up dosing schedule (thereby resuming the planned dosing regimen), or
(v) if the last dose administered to the subject was 0.4 mg/kg, and the time from the last dose administered is 29 to 56 days, then restarting the step-up dosing schedule at the second step-up dose of 0.06 mg/kg and resuming the planned dosing regimen, or
(vi) if the last dose administered to the subject was 0.4 mg/kg, and the time from the last dose administered is more than 56 days, then either discontinuing the planned dosing regimen permanently, or restarting the step-up dosing schedule at the first step-up dose of 0.01 mg/kg and resuming the planned dosing regimen, or
(vii) if the last dose administered to the subject was 0.8 mg/kg, and the time from the last dose administered is 15 to 28 days, then continuing the planned dosing regimen at treatment doses of 0.8 mg/kg every two weeks (thereby resuming the planned dosing regimen); or
(viii) if the last dose administered to the subject was 0.8 mg/kg, and the time from the last dose administered is 29 to 56 days, then restarting the step-up dosing schedule at the third step-up dose of 0.4 mg/kg and resuming the planned dosing regimen, or
(ix) if the last dose administered to the subject was 0.8 mg/kg, and the time from the last dose administered is more than 56 days, then either discontinuing the planned dosing regimen permanently, or restarting the step-up dosing schedule at the first step-up dose of 0.01 mg/kg and resuming the planned dosing regimen.

45. The method of claim 44, wherein the step-up dosing schedule includes (a) the first step-up dose of 0.01 mg/kg on Day 1, (b) the second step-up dose of 0.06 mg/kg on Day 4, (c) the third step-up dose of 0.4 mg/kg on Day 7, and (d) the first treatment dose of 0.8 mg/kg on Day 10.

46. The method of claim 44, wherein the step-up dosing schedule includes (a) the first step-up dose of 0.01 mg/kg on Day 1, (b) the second step-up dose of 0.06 mg/kg on Day 3, (c) the third step-up dose of 0.4 mg/kg on Day 5, and (d) the first treatment dose of 0.8 mg/kg on Day 7.

47. The method of claim 44, wherein each of the second step-up dose and the third step-up dose is administered between 2 to 4 days after the previous dose, or is optionally administered up to 7 days after the previous dose to allow for resolution of adverse reactions; and the first treatment dose is administered between 2-7 days after the third step-up dose.

48. The method of claim 44, wherein each of the second step-up dose, the third step-up dose and the first treatment dose is administered between 2 to 4 days after the previous dose, or is optionally administered up to 7 days after the previous dose to allow for resolution of adverse reactions.

49. The method of claim 40 further comprising administering pretreatment medications to the subject prior to restarting the planned dosing regimen, wherein the pretreatment medications comprise 16 mg or equivalent of oral or intravenous dexamethasone, 50 mg or equivalent of oral or intravenous diphenhydramine, and 650 mg to 1000 mg, or equivalent, oral or intravenous acetaminophen.

50. The method of claim 40, wherein the GPRC5DxCD3 bispecific antibody comprises a GPRC5D binding domain comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6, and a CD3 binding domain comprising the HCDR1 of SEQ ID NO: 11, the HCDR2 of SEQ ID NO: 12, the HCDR3 of SEQ ID NO: 13, the LCDR1 of SEQ ID NO: 14, the LCDR2 of SEQ ID NO: 15 and the LCDR3 of SEQ ID NO: 16.

51. The method of claim 40, wherein the GPRC5DxCD3 bispecific antibody comprises a GPRC5D binding domain comprising a heavy chain variable region (VH) having the amino acid sequence of SEQ ID NO: 7 and a light chain variable region (VL) having the amino acid sequence of SEQ ID NO: 8, and the CD3 binding domain comprises a heavy chain variable region (VH) having the amino acid sequence of SEQ ID NO: 17 and a light chain variable region (VL) having the amino acid sequence of SEQ ID NO: 18.

52. The method of claim 40, wherein the GPRC5DxCD3 bispecific antibody is an IgG1, an IgG2, an IgG3 or an IgG4 isotype.

53. The method of claim 40, wherein the GPRC5DxCD3 bispecific antibody is an IgG4 isotype.

54. The method of claim 40, wherein the GPRC5DxCD3 bispecific antibody comprises one or more substitutions in its Fc region.

55. The method of claim 40, wherein the GPRC5DxCD3 bispecific antibody is an IgG4 isotype and comprises Proline/Alanine/Alanine substitutions at amino acid positions 228/234/235, respectively, in its Fc region (according to EU index numbering).

56. The method of claim 40, wherein the GPRC5DxCD3 bispecific antibody is an IgG4 isotype and comprises F405L and R409K substitutions in its Fc region (according to EU index numbering).

57. The method of claim 40, wherein the Fc region of the GPRC5D-binding arm comprises Proline/Alanine/Alanine substitutions at amino acid positions 228/234/235, respectively (according to EU index numbering).

58. The method of claim 40, wherein the Fc region of the CD3-binding arm comprises Proline/Alanine/Alanine substitutions at amino acid positions 228/234/235, respectively, in addition to F405L and R409K substitutions (according to EU index numbering).

59. The method of claim 40, wherein the GPRC5DxCD3 bispecific antibody comprises a first heavy chain (HC1) having the amino acid sequence of SEQ ID NO: 9, a first light chain (LC1) having the amino acid sequence of SEQ ID NO: 10, a second heavy chain (HC2) having the amino acid sequence of SEQ ID NO: 19 and a second light chain (LC2) having the amino acid sequence of SEQ ID NO: 20.

60. The method of claim 40, wherein the GPRC5DxCD3 bispecific antibody comprises a first heavy chain (HC1) having at least 90% identity to the amino acid sequence of SEQ ID NO: 9, a first light chain (LC1) having at least 90% identity to the amino acid sequence of SEQ ID NO: 10, a second heavy chain (HC2) having at least 90% identity to the amino acid sequence of SEQ ID NO: 19 and a second light chain (LC2) having at least 90% identity to the amino acid sequence of SEQ ID NO: 20.

61. The method of claim 40, wherein the GPRC5DxCD3 bispecific antibody comprises a first heavy chain (HC1) having at least 95% identity to the amino acid sequence of SEQ ID NO: 9, a first light chain (LC1) having at least 95% identity to the amino acid sequence of SEQ ID NO: 10, a second heavy chain (HC2) having at least 95% identity to the amino acid sequence of SEQ ID NO: 19 and a second light chain (LC2) having at least 95% identity to the amino acid sequence of SEQ ID NO: 20.

62. The method of claim 40, wherein the GPRC5DxCD3 bispecific antibody comprises a first heavy chain (HC1) having at least 98% identity to the amino acid sequence of SEQ ID NO: 9, a first light chain (LC1) having at least 98% identity to the amino acid sequence of SEQ ID NO: 10, a second heavy chain (HC2) having at least 98% identity to the amino acid sequence of SEQ ID NO: 19 and a second light chain (LC2) having at least 98% identity to the amino acid sequence of SEQ ID NO: 20.

63. The method of claim 40, wherein the GPRC5DxCD3 bispecific antibody comprises a first heavy chain (HC1) having at least 99% identity to the amino acid sequence of SEQ ID NO: 9, a first light chain (LC1) having at least 99% identity to the amino acid sequence of SEQ ID NO: 10, a second heavy chain (HC2) having at least 99% identity to the amino acid sequence of SEQ ID NO: 19 and a second light chain (LC2) having at least 99% identity to the amino acid sequence of SEQ ID NO: 20.

64. The method of claim 40, wherein the GPRC5DxCD3 bispecific antibody is talquetamab.

65. The method of claim 40, wherein the subject has relapsed or refractory multiple myeloma.

66. The method of claim 40, wherein the subject has relapsed and refractory multiple myeloma and has received at least 3 prior therapies, including an immunomodulatory agent, a proteasome inhibitor, and an anti-CD38 antibody and has demonstrated disease progression on the last therapy.

67. The method of claim 40, wherein the subject has relapsed or refractory multiple myeloma and has received at least four prior lines of therapy, including a proteasome inhibitor, an immunomodulatory agent and an anti-CD38 monoclonal antibody.


SEQUENCE LISTING (20 sequences)

SEQ ID NO: 1 - protein, 5 aa, synthetic construct
GYTMN

SEQ ID NO: 2 - protein, 17 aa, synthetic construct
LINPYNSDTNYAQKLQG

SEQ ID NO: 3 - protein, 9 aa, synthetic construct
VALRVALDY

SEQ ID NO: 4 - protein, 11 aa, synthetic construct
KASQNVATHVG

SEQ ID NO: 5 - protein, 7 aa, synthetic construct
SASYRYS

SEQ ID NO: 6 - protein, 9 aa, synthetic construct
QQYNRYPYT

SEQ ID NO: 7 - protein, 118 aa, synthetic construct
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS

SEQ ID NO: 8 - protein, 107 aa, synthetic construct
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIK

SEQ ID NO: 9 - protein, 445 aa, synthetic construct
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
TYAMN

SEQ ID NO: 12 - protein, 19 aa, synthetic construct
RIRSKYNNYATYYAASVKG

SEQ ID NO: 13 - protein, 14 aa, synthetic construct
HGNFGNSYVSWFAY

SEQ ID NO: 14 - protein, 14 aa, synthetic construct
RSSTGAVTTSNYAN

SEQ ID NO: 15 - protein, 7 aa, synthetic construct
GTNKRAP

SEQ ID NO: 16 - protein, 9 aa, synthetic construct
ALWYSNLWV

SEQ ID NO: 17 - protein, 125 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 18 - protein, 112 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQP

SEQ ID NO: 19 - protein, 452 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 20 - protein, 215 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS